(19) United States
(12) Patent Application Publication
(10) Pub. No.: US 2007/0054300 A1
(43) Pub. Date: Mar. 8, 2007

(54) Method for the identification and quantification of microorganisms useful in biomining processes

(75) Inventors: Pilar Angelica Parada Valdecantos, Nunoa (CL); Katia Nicole Ehrenfeld Stolzenbach, Las Condes (CL); Igor Alejandro Pacheco Cruz, Recoleta (CL); Alejandro Eduardo Maass Sepulveda, Penalolen (CL); Andres Octavio Aravena Duarte, Nunoa (CL); Mauricio Alejandro Gonzalez Canales, Penalolen (CL); Servet Martinez Aguilera, La Cisterna (CL)

Correspondence Address: MERCHANT & GOULD PC, P.O. BOX 2903, MINNEAPOLIS, MN, 55402-0903, US

(73) Assignee: BIOSIGMA S.A., Colina (CL)

(21) Appl. No.: 11/509,870

(22) Filed: Aug. 25, 2006

(30) Foreign Application Priority Data
Aug. 26, 2005 (CL) 2179-2005

(51) Int. Cl. C12Q 1/68; G06F 19/00

(52) U.S. Cl. 435/6; 702/20

(57) ABSTRACT

The present invention discloses a method to identify and quantify environmental microorganisms useful in biomining processes. These microorganisms are basically 10, belonging to Bacteria: Acidiphilium sp., Leptospirillum sp., Sulfobacillus sp., Acidithiobacillus ferrooxidans and Acidithiobacillus thiooxidans; and Archaea: Acidianus sp., Ferroplasma sp., Metallosphaera sp., Sulfolobus sp. and Thermoplasma sp.

The method comprises performing a two-stage PCR known as nested PCR, where in the first stage, called primary PCR, 16S ribosomal DNA sequences (nucleotides 27 to 1492, with E. coli rDNA numbering) are amplified using universal primers for the Bacteria and Archaea kingdoms. In the second stage, these primary amplicons are used as template in qPCR reactions, called secondary PCR, in which internal universal primers for either Bacteria or Archaea kingdoms, as it corresponds, and specific primers designed in our laboratories for different taxons to be determined are used. The first PCR linearly multiplies 16S sequences from bacteria or archaea, thus increasing template abundance for the secondary PCR keeping the original microorganism proportion of the sample. This gives a higher sensitivity to the process when compared to the case of directly using taxon-specific primers on the sample. With qPCR results and other data obtained from the analyzed sample, the microorganism concentration of each analyzed taxon present in the sample is calculated using a mathematical formula.

### FIELD OF THE INVENTION

The present invention discloses a method to identify and quantify microorganisms useful in biomining processes that are present in a given sample. This method is presented as a useful tool in biomining, in every case where the present microbiological population needs to be evaluated, whether on the mineral, in solutions, in bioleaching heaps, in biomining laboratories or in any other circumstance that involves the use of such microorganisms.

### BACKGROUND OF THE INVENTION

Biomining is, in general terms, the use of microorganisms for metal recovery from mineral ores. Its most traditional expression is bioleaching, but not only this process is understood as biomining, but also the monitoring and intervention in such process, as these techniques are complex and are under constant development; and also laboratory research associated to process improvement or the development of new methodologies.

Until now, bioleaching continues to be the most important process in biomining field, and is defined as a method to solubilize metals from complex matrixes in an acid medium, using direct or indirect microorganism action. The microorganisms that are useful in these processes belong to Bacteria or Archaea kingdoms, and fulfill two basic conditions: they are acidophilic and chemolithotrophic.

### Microbiological Diversity in Communities Associated to Bioleaching Processes

Various microorganisms have been described to be useful in bioleaching processes, and ten taxons could be identified among them: 3 genera and 2 species from the Bacteria kingdom, namely Acidiphilium sp., Leptospirillum sp., Sulfobacillus sp. genera and Acidithiobacillus ferrooxidans and Acidithiobacillus thiooxidans species, and five genera from the Archaea kingdom, namely Acidianus sp., Ferroplasma sp., Metallosphaera sp., Sulfolobus sp. and Thermoplasma sp. (Rawlings D E. Heavy metal mining using microbes. Annu Rev Microbiol. 2002; 56:65-91; Rawlings D E. Characteristics and adaptability of iron- and sulfur-oxidizing microorganisms used for the recovery of metals from minerals and their concentrates. Microb Cell Fact. May 6, 2005; 4(1):13).

### Factors Determining Diversity and Metabolic Activity of the Microbial Community Associated to a Bioleaching Process

Each of the above mentioned genera or species catalyzes different reactions and require in its turn different conditions to perform such reaction, which could be, for instance, aerobic or anaerobic, or could require some specific nutrient. Therefore, the environmental conditions in which a bioleaching process is performed will modify the bacterial composition of the community.

Additionally, the participation of microorganisms in a bioleaching process has been proposed to be direct and/or indirect (Rawlings D E. Characteristics and adaptability of iron- and sulfur-oxidizing microorganisms used for the recovery of metals from minerals and their concentrates. Microb Cell Fact. May 6, 2005; 4(1):13). When the action is direct, microorganisms directly oxidize the target metal or its counter-ion, in both cases liberating into the solution a target metal ion. On the other hand, when the action is indirect, the substrate of the microorganism is not the target metal neither its counter-ion, but instead chemical conditions are generated that allow the solubilization of said metal, either by acidification of the medium (e.g., by generating sulfuric acid) or by the generation of an oxidizing agent that ultimately interacts with the salt (metal and counter-ion) to be solubilized.

Regarding this aspect, it is possible that the bacterial community changes its species composition as a function of the bioleaching type being performed in different mineral samples and/or the environmental conditions in which this process is carried out.

For instance, Acidithiobacillus species are able to catalyze the oxidation of reduced sulfur compounds (e.g., sulfide, elemental sulfur, thionates, etc.) using oxygen as electronic acceptor and generating sulfuric acid as final product and reducing species like sulfite and thiosulfate as intermediate products, which allows the solubilization of metals associated to sulfides in the mineral. Acidithiobacillus ferrooxidans and Leptospirillum ferrooxidans are able to catalyze the oxidation of iron(II) to iron(III) using oxygen as electron acceptor, being the generated iron(III) a great oxidizing agent that can oxidize sulfides in the mineral or any other compound to be oxidized.

The usual mining practice in bioleaching processes is to leave a mineral heap in an acid medium, generally sulfuric acid, and constantly remove the acid medium to recover the metal by electrolysis. Usually heaps in which the recovery yield of the metal is efficient are obtained, and also “inefficient” heaps that have a low yield under the same operation conditions and characteristics of the substrate to be leached. The explanation to this unequal result requires the elucidation of differences in abundance and types of species in the microbiological community between both heaps. In this way, the low yield problem could be explained by the microbial community composition, and could be solved in its turn by inoculation of microorganisms that catalyze the reaction to be maintained during the bioleaching process. However, a method that enables to quantify the population of archaea and bacteria useful in biomining processes is not available up to this date. In this patent, a method is described that solves the technical problem previously described, by designing a method to identify and quantify the presence of known microorganisms that are most relevant in biomining processes, namely the bacteria: Acidiphilium sp., Leptospirillum sp., Sulfobacillus sp., Acidithiobacillus ferrooxidans and Acidithiobacillus thiooxidans; and the archaea: Acidianus sp., Ferroplasma sp., Metallosphaera sp., Sulfolobus sp. and Thermoplasma sp.

Nested polymerase chain reaction (PCR) was the technique selected to develop this method. In this technique, a conserved genome region of the microorganisms is firstly amplified in a first PCR reaction, either on bacteria or archaea. We have selected gene 16SrDNA as the conserved region. Then, taxon-specific primers (targeting genera or species) are used to identify the presence of target microorganisms in a second PCR reaction. This second PCR reaction is performed using an equipment that allows measuring the increase of amplified product in each amplification cycle, and this information allows the quantification, by interpolation, of the original abundance of the target genome in the sample being analyzed. PCR reaction under these conditions is called quantitative PCR or qPCR.

A critical step in nested PCR technique is the design of primers for the second amplification reaction, which have to be specific for the taxon to be determined, and this aspect has a vital importance in this particular case, as the samples to which the process will be applied will usually be metagenomic samples. Therefore, it is necessary to reduce the possibility of primer unspecific hybridization to sequences present in the genome of microorganisms that have not yet been identified in the community. We have generated two fundamental tools for the design of these primers: firstly, a depurated 16SrDNA sequence database obtained from all disclosed 16SrDNA sequences; and a computational program for primer design that uses as input such database and allows designing thermodynamically stable taxon specific primers.

In the state of the art there are many examples of the application of nested PCR or qPCR, but none of them is focused to bacteria or archaea useful in biomining processes. For instance, J. L. M. Rodrigues et al (Journal of Microbiological Methods 51 (2002) 181-189) describe a qPCR to detect and quantify PCB-degrading Rhodococcus present in soil, where the 16SrDNA gene belonging to the strain with the target activity is sequenced, specific primers for said sequence are designed and qPCR reactions are carried out using said primers. In this document, a direct qPCR approach is used, instead of a nested qPCR, and it is directed to other type of microorganisms, whose handling has been widely studied and many techniques for DNA extraction are available. Another document that uses a similar approach is Patent Application EP 1 484 416, which discloses a method for the detection and quantification of pathogen bacteria and fungi present in an environment sample using qPCR. The method comprises the extraction of DNA from bacteria and fungi present in an environment sample, obtaining specific sense and antisense primers for each of the taxons to be detected and quantified; and performing qPCR reactions using a pair of primers for each of the target pathogens.

Although it is possible to enumerate documents in which microorganisms are identified and quantified using quantitative PCR techniques, as they are well known techniques in the art, the relevant point is the generation of a depurated database that allows to design specific primers and has not been implemented before for the identification of microorganisms useful in biomining processes, which is subject matter of this invention.

### SUMMARY OF THE INVENTION

The present invention discloses a method to identify and quantify environmental microorganisms useful in biomining processes. These microorganisms are basically 10, belonging to Bacteria: Acidiphilium sp., Leptospirillum sp., Sulfobacillus sp., Acidithiobacillus ferrooxidans and Acidithiobacillus thiooxidans; and Archaea: Acidianus sp., Ferroplasma sp., Metallosphaera sp., Sulfolobus sp. and Thermoplasma sp.

The method comprises performing a two-stage PCR known as nested PCR, where in the first stage, called primary PCR, 16S ribosomal DNA sequences (nucleotides 27 to 1492, with E. coli rDNA numbering) are amplified using universal primers for the Bacteria and Archaea kingdoms. In the second stage, these primary amplicons are used as template in qPCR reactions, called secondary PCR, in which internal universal primers for either Bacteria or Archaea kingdoms, as it corresponds, and specific primers designed in our laboratories for different taxons to be determined are used. The first PCR linearly multiplies 16S sequences from bacteria or archaea, thus increasing template abundance for the secondary PCR keeping the original microorganism proportion of the sample. This gives a higher sensitivity to the process when compared to the case of directly using taxon-specific primers on the sample. However, the method also works and is applicable without the primary PCR, and therefore this stage may be optional.

With qPCR results and other data obtained from the analyzed sample, the microorganism concentration of each analyzed taxon present in the sample is calculated using a mathematical formula.

### BRIEF DESCRIPTION OF THE DRAWINGS

FIGS. 1 to 7 show the results of Example 1, where the presence of Acidithiobacillus thiooxidans, Acidithiobacillus ferrooxidans, Leptospirillum sp., Acidiphilium sp. and total bacteria has been quantified in 7 different samples. In each Figure, results for each solid sample (MS), identified as MS-1, MS-2, MS-3, MS-4 and MS-5, and each liquid sample (ML), identified as ML-1 and ML-2, are plotted. Each plot shows quantified taxons in the abscissa and microorganism number per sample unit in logarithmic scale in the ordinate. Data giving origin to plots are shown in Tables 26 to 32.

From these results it is possible to observe which one is the predominant species in each of the mineral samples from bioleaching heaps (MS-1 to MS-5) and from liquid samples recovered from bioleaching effluents (ML-1 and ML-2). This value can also be correlated to total bacteria found in said samples. Thus, in 6 over 7 samples Leptospirillum sp. predominance is observed, and even more, only this microorganism genus is present in one of the liquid samples. Only one of the solid samples (MS-2) shows A. thiooxidans predominance, which leads to the conclusion that Leptospirillum sp. is the most abundant microorganism in this type of mineral samples.

FIGS. 8 and 9 show the results of Example 2, where the presence of Sulfobacillus sp, Sulfolobus sp, Ferroplasma sp., total bacteria and total archaea was quantified on 2 different samples obtained from bioleaching heap mineral. In each Figure, results for each solid sample (MS), identified as MS-6 and MS-7 are plotted. Each plot shows quantified taxons in the abscissa and microorganism number per sample unit in logarithmic scale in the ordinate. Data used for generating these plots are shown in Tables 45 and 46. From these results it can be concluded that the presence of microorganisms belonging to the Archaea kingdom is minority in both samples, compared to those belonging to the Bacteria kingdom. However, specific determinations of the genus Sulfolobus (archaea) in sample MS-6 are slightly higher than the number of bacteria belonging to the genus Sulfobacillus, which indicates the presence of a high number of bacteria from other genera in the sample. Likewise, a microorganism belonging to the genus Ferroplasma is detected in sample MS-7, and it is absent in the former sample. Again, these data could give an explanation to specific behaviors of the community that is present in the analyzed mineral.

### DETAILED DESCRIPTION OF THE INVENTION

As has been anticipated, the invention relates to a method that allows the identification and quantification of essential microorganisms in biomining processes. These essential microorganisms belong to 10 taxons, the genera Acidiphilium sp., Leptospirillum sp., Sulfobacillus sp. and the species Acidithiobacillus ferrooxidans and Acidithiobacillus thiooxidans belonging to the Bacteria kingdom; and the genera Acidianus sp., Ferroplasma sp, Metallosphaera sp, Sulfolobus sp. and Thermoplasma sp. belonging to the Archaea kingdom.

As previously indicated, a method to identify and quantify biomining microorganisms would have applications in different industrial tasks and areas. For instance, a good tool for suitable control of bioleaching process could be the identification of microorganisms that are present in a bioleaching heap and how abundant they are, as it could be established whether is necessary to inoculate some particular microorganism or simply determine which nutrients should be added to the mixture, thus maximizing the quantity of mineral recovered in the process. The idea is to correlate the recovery efficiency of different metals present in the heap with the composition of the microbiological community in the heap, referred to the number and type of present individuals.

In general terms, samples to be analyzed in the method of the invention will be biomining samples, but this does not limit the scope of the invention, as the described method could be applied any time that one or more of the 10 taxons subject of this invention is to be identified and quantified.

In the description of the invention, all oligonucleotide sequences are written in direction 5′ to 3′. Described oligonucleotides correspond to primers for PCR reactions, which can be sense or antisense primers, which could be indicated specifically (e.g., as table titles) or alternatively by including letter “F” for sense or forward primers and “R” for antisense or reverse primers in the name of the primer.

The following is the description of each of the stages of the method in detail.

DNA Preparation.

In a first stage, it is necessary to extract DNA from the sample. Different methods to extract DNA from mineral or soil samples have been disclosed and any of them can be used, considering in each case the particular nature of the sample (Appl Environ Microbiol. July 2003; 69(7):4183-9; Biotechniques. April 2005; 38(4):579-86). In the case that total extracted DNA (from mineral samples, being e.g. grounded chalcopyrite type 1 or other) is turbid or has a yellow or orange color, it is recommended to repurify the sample using any existing purification technique; in our laboratories this step is performed using commercial DNA purification columns. The purified fraction is resuspended in sterile nuclease-free H₂O.

Once total DNA samples have been purified, total DNA present in the sample should be quantified; again, this quantification could be performed using any existing method to quantify DNA; in our laboratories it is done by spectrophotometry.

After quantifying total DNA present in the sample, an aliquot is taken and diluted to a concentration suitable for the method, which finally ranges from 0.5 to 40 ng/μl, preferably from 1 to 30 ng/μl, and most preferably from 1 to 10 ng/μl. The dilution must be done using sterile nuclease-free water.

Primary PCR.

This stage is optional and could be skipped, however in our case it is advantageous to perform it as it restrict the analyzed subject universe and increases the sensitivity of the method. Using the DNA sample previously prepared at least one of the primary PCR is performed, one of them using primers to amplify 16S region from the Bacteria kingdom (“universal Bacteria primers”) and the other using primers to amplify 16S region from the Archaea kingdom (“universal Archaea primers”).

Primary PCRs are intended to amplify the region coding for 16S ribosomal RNA, for which any primer pair could be used in primary PCR that fulfill the described requirements; in our laboratories universal primers shown in the list included in Table 1 are preferentially selected.

TABLE 1
Primary PCR

Bacteria primers

| | |
|---|---|
| Eub27-F¹ | AGA GTT TGA TCC TGG CTC AG |
| Univ1492-R¹ | GGT TAC CTT GTT ACG ACT T |

Archaea primers

| | |
|---|---|
| Arch21-F² | TTC CGG TTG ATC C(CT)G CCG GA |
| Univ1492-R¹ | GGT TAC CTT GTT ACG ACT T |

¹Bond P., 2000, Appl Environ Microbiol. 66(9):3842-9.
²Delong, E.F., 1992, Proc. Nac. Acad. Sci. USA 89: 5685-9.

It is important that primary PCR should be linear, i.e., amplification does not reach saturation, as the original proportion in the sample should be kept.

This PCR is also applied on a negative control, containing sterile water instead of DNA, and a five-point calibration curve, formed by a master mix and four serial dilutions thereof. The master mix is specific for each kingdom, Bacteria or Archaea, and is formed by a standard DNA mix belonging to each of the taxons to be determined. This means that in the PCR using universal Bacteria primers, the standard DNA mix used in the master mix will contain pure DNA extracted from all bacteria to be identified and quantified, as Acidiphilium sp., Leptospirillum sp., Sulfobacillus sp., Acidithiobacillus ferrooxidans and Acidithiobacillus thiooxidans; whereas in the primary PCR using universal Archaea primers the master mix will contain DNA from all Archaea to be identified and quantified, as Acidianus sp., Ferroplasma sp, Metallosphaera sp, Sulfolobus sp. and Thermoplasma sp.

The master mix optimally contains from 1 to 100 ng of DNA from each of the strains, and preferably contains 100 ng of total DNA, although it is possible to work with higher or lower amounts. The calibration curve will be used in the quantification that will be performed with secondary PCR and corresponds to the master mix in concentrations 1×, 0.1×, 0.01×, 0.001× and 0.0001×. Each one of these dilutions is subjected to the primary PCR.

Preferably, each primary PCR is carried out using 1 μl of DNA, either for the sample or the calibration curve, or 1 μl of water for the negative control, plus 24 μl of the reaction mix whose composition is described in Table 2.

TABLE 2

| | |
|---|---|
| Sterile nuclease-free H₂O | 18.35 μl |
| PCR Buffer 10x | 2.5 μl |
| MgCl₂ (50 mM) | 1.5 μl |
| dNTPs (10 mM each) | 0.5 μl |
| Primer Eubac27F (10 μM) | 0.5 μl |
| Primer Univ1492R (10 μM) | 0.5 μl |
| Hot Start Taq (5 U/μl) | 0.15 μl |

Primary PCR cycles are described in Table 3.

TABLE 3

| | Temperature | |
|---|---|---|
| Step | (° C.) | Time (s) |
| 1. Initial denaturation | 95 | 120 |
| 2. Denaturation | 95 | 30 |
| 3. Alignment | | |
| 3.1. for Bacteria | 56 | 30 |
| 3.2. for Archaea | 57 | 30 |
| 4. Extension | 72 | 120 |

Wherein steps 2 to 4 are repeated from 15 to 20 times, avoiding saturation.

Once this primary PCR has been performed, the sequence of region 16S of all bacteria and archaea present in the original sample has been amplified.

Secondary PCR.

Then, a plurality of PCR is carried out, specific for each taxon to be identified, using specific primers that amplify inside the 16S rDNA region amplified in the primary PCR.

In this stage it is crucial to have specific and efficient primers to amplify the target fragment that have no cross-reaction with organisms from other taxons and are thermodynamically stable, i.e. do not form hairpins, homodimers or heterodimers. The primers used in this application have been designed using the method disclosed in Patent Application CL 2102-2005 filled by Biosigma; as said method guarantees the efficiency and specificity of the designed primers.

In each primary PCR a reaction has been carried out to amplify each of the samples, 5 point of the calibration curve and one negative control. Each secondary PCR will be performed on all the reaction products of each corresponding primary PCR reaction. Advantageously, all reactions are carried out in duplicate, and a negative control is added.

When we say that secondary PCR is performed on the corresponding primary PCR, we mean that if our target taxon to be amplified in the secondary PCR belongs to the Archaea kingdom, then we will use the primary PCR reaction products for archaea. Likewise, if the taxon to be quantified is a bacterium, we will use the primary PCR reaction products for bacteria in the secondary PCR.

It is important to point out that the method of the invention can be carried out to identify and quantify either all the described taxons or only one of them, and also all the possible intermediate combinations, and as a consequence every one of these options will remain being comprised inside the scope of the present invention.

The secondary PCR is a quantitative PCR (qPCR), therefore it should be performed in a suitable thermocycler and using fluorescent reagents for qPCR. There are different commercially available alternatives, either for equipment or reagents, and any of them can be selected to carry out the present method.

For each secondary PCR reaction the following mix is prepared:

TABLE 4

| | |
|---|---|
| Sterile nuclease-free H₂O | 10.5 μl |
| Sense primer (10 μM) | 0.5 μl |
| Antisense primer (10 μM) | 0.5 μl |
| qPCR reagent | 12.5 μl |

To the mix described in Table 4, 1 μl of primary PCR reaction product or sterile water for the qPCR blank is added.

### Primers for the Secondary PCR

As previously indicated, the requirements to be fulfilled by each primer pair selected for the secondary PCR are: being specific for each taxon, having no cross-reactivity and being thermodynamically stable to assure primer availability in the PCR reaction. Our laboratory has developed a primer design program that gives a large amount of primers fulfilling these requirements. The method of the invention can be performed by combining any sense primer with any antisense primer designed by our program. In following tables, we give 20 sense primers and 20 antisense primers for each taxon, where any possible combination thereof could be selected for the qPCR. (Note: the sequences of the designed primers have been compared, by using Blast from NCBI, with previously existent sequence disclosures, thus guaranteeing its novelty as primers.)

Bacteria Kingdom:

TABLE 5
Acidiphilium sp.

| | |
|---|---|
| Sense primers | Antisense primers |

| | |
|---|---|
| CAA CCA CGG TCG GGT CAG | TCT CTG ACC CGA CCG TGG |
| A | TT |
| GAC CTT AAG TTG ATG CGC | TCA ACT TAA GGT CAA ACC |
| T | AA |
| AGT CAA CCA CGG TCG GGT | GGA GCT TAT TCT GCG GGT |
| C | A |
| GGT TTG ACC TTA AGT TGA | GCA TCA ACT TAA GGT CAA |
| TG | AC |
| CTT AAG TTG ATG CGC TAA | AGC GCA TCA ACT TAA GGT |
| C | CA |
| GGC AGT CAA CCA CGG TCG | GTT AGC GCA TCA ACT TAA |
| G | GG |
| CGA TGC TGA GCT GAT CCT | CCG ACC GTG GTT GAC TGC |
| G | C |
| AAG TTG ATG CGC TAA CCG | GGA TCA GCT CAG CAT CGC |
| C | TG |
| AAA GTC GCC TAA GGA GGA | TCA GGA TCA GCT CAG CAT |
| G | CG |
| GTC GCC TAA GGA GGA GCC | CGG TTA GCG CAT CAA CTT |
| T | A |
| AAG GAG GAG CCT GCG TCT | GGC TCC TCC TTA GGC GAC |
| G | TT |
| AGG AGC CTG CGT CTG ATT | GTT GAC TGC CTC CTT GCG |
| A | GT |
| AGG AGG CAG TCA ACC ACG | TCC TCC TTA GGC GAC TTT |
| GT | CG |
| GCG AAA GTC GCC TAA GGA | GTG GTT GAC TGC CTC CTT |
| G | GC |
| GCC TAA GGA GGA GCC TGC | ACC GTG GTT GAC TGC CTC |
| GT | CT |
| GCA AGG AGG CAG TCA ACC | GCA GGC TCC TCC TTA GGC |
| A | GA |
| GCA AGT CGC TCG GGC AGT | GAC GCA GGC TCC TCC TTA |
| A | GG |
| ACC CGT AGG AAT CTA TCC | TCA GAC GCA GGC TCC TCC |
| T | TT |
| GCA CAG TCA GGC GTG AAA | TGC TAC TGC CCG AGC GAC |
| TA | TT |
| ACA CAT GCA AGT CGC TCG | TGA CCC GAC CGT GGT TGA |
| GG | C |

TABLE 6
Leptospirillum sp.

| | |
|---|---|
| Sense primers | Antisense primers |

| | |
|---|---|
| TGA GGG GAC TGC CAG CGA | CTA GAC GGG TAC CTT GTT |
| C | AC |
| TAA ATA TCC CCG ATG ACG | CCG TCA TCG GGG ATA TTT |
| G | A |
| TTG TCC GGA ACC GTG AAG | TTC ACG GTT CCG GAC AAT |
| GG | AT |
| GGA ACC GTG AAG GGT TTC | CGG TTC CGG ACA ATA TTC |
| G | G |
| CCG AAT ATT GTC CGG AAC | CCC TTC ACG GTT CCG GAC |
| C | AA |
| CGA CAG AGT TTG ATC GTG | CCA CGA TCA AAC TCT GTC |
| G | GA |
| AAT ATT GTC CGG AAC CGT | AAA CCC TTC ACG GTT CCG |
| G | GA |
| TCC GGA ACC GTG AAG GGT | TTC CGG ACA ATA TTC GGT |
| T | AT |
| AAA TCG GGC CAT CAC ACA | CCG AAA CCC TTC ACG GTT |
| G | CC |
| CAA AGA GAC TGG CAG ACT | TAG TCT GCC AGT CTC TTT |
| AGA | GGC |
| TCG GGC CAT CAC ACA GGT | GCA CCT GTG TGA TGG CCC |
| G | GAT |
| AGA GAC TGG CAG ACT AGA | CTC TAG TCT GCC AGT CTC |
| G | TTT |
| GGG GGG GCA ATA CCG AAT | GCA GCA CCT GTG TGA TGG |
| AGA | CCC |
| ATA TCA AAT AAA TAT CCC | CCT GTG TGA TGG CCC GAT |
| CG | TT |
| AAG GGA TAT CGA ATA AAT | TCT ATT CGG TAT TGC CCC |
| AT | CCC |
| CTA GAG GCT GGG AGA GGG | CCC CTT TCG GTT CCC TAC |
| AAG | TCG |
| GAC GCA GCA ACG CCA GCA | TCC CTC TCC CAG CCT CTA |
| GTG | GTC |
| AAA TAA ATA TCC CCG ATG | TCG GGG ATA TTT ATT TGA |
| A | T |
| CAG TGT GGG AAG AAG GCT | CAT ACC TTG GGC GGC TCC |
| TTC | CT |
| AAC AAG GTA CCC GTC TAG | CAG CCT CTA GTC TGC CAG |
| A | T |

TABLE 7
Sulfobacillus sp.

| | |
|---|---|
| Sense primers | Antisense primers |

| | |
|---|---|
| CGA AGG CGG TGC ACT GGC | CAG TGC ACC GCC TTC GCC |
| C | A |
| GTG GCG AAG GCG GTG CAC | GGC CAG TGC ACC GCC TTC |
| T | G |
| AGG TGT CGC GGG GGT CCA | GGT GGA CCC CCG CGA CAC |
| CC | C |
| TGT CTG TCG GGA CGA GGA | GGT CCT CGT CCC GAC AGA |
| C | C |
| GAG GGC AGG AGA GGT GCA | CAT GCA CCT CTC CTG CCC |
| T | TC |
| GTC CAC CTC GCG GTG CCG | TTA GCT CCG GCA CCG CGA |
| G | GG |
| CAC CTC GCG GTG CCG GAG | GCG AGG TGG ACC CCC GCG |
| C | A |
| GGG GGT CCA CCT CGC GGT | TGC ACC GCC TTC GCC ACC |
| GC | G |
| CTC GCG GTG CCG GAG CTA | CGT ATC CAT CGT TTA CGG |
| A | CG |
| TGT CGC GGG GGT CCA CCT | GAC CCC CGC GAC ACC TCG |
| C | TA |
| GGA TAC GAG GTG TCG CGG | GAG TGC GTT AGC TCC GGC |
| G | AC |
| CGG AGC TAA CGC ACT CAG | TCC ACC AGG AAT TCC ATG |
| T | C |
| GTA AAC GAT GGA TAC GAG | GCC AGG CCA GTG CAC CGC |
| GT | C |
| TGA GTG GGG GAT ATC GGG | CCA GGA ATT CCA TGC ACC |
| C | TC |
| TAC GAG GTG TCG CGG GGG | CCT CGT ATC CAT CGT TTA |
| T | CG |
| AGC TAA CGC ACT CAG TAT | ACT GAG TGC GTT AGC TCC |
| C | GG |
| ACG ATG GAT ACG AGG TGT | GAT ACT GAG TGC GTT AGC |
| CG | TC |
| GTG CCG GAG CTA ACG CAC | GCG ACA CCT CGT ATC CAT |
| TC | CG |
| AGG TGC ATG GAA TTC CTG | CGG GAT ACT GAG TGC GTT |
| GT | AG |
| TGC ATG GAA TTC CTG GTG | GCC CGA TAT CCC CCA CTC |
| GA | A |

TABLE 8
Acidithiobacillus ferrooxidans

| | |
|---|---|
| Sense primers | Antisense primers |

| | |
|---|---|
| CGG GTT CTA ATA CAA TCT | AGA ACC CGC CTT TTC GTC |
| G | CT |
| AGG ACG AAA AGG CGG GTT | CCG CCT TTT CGT CCT CCA |
| CT | C |
| GTG GAG GAC GAA AAG GCG | CAG ATT GTA TTA GAA CCC |
| G | G |
| ACG AAA AGG CGG GTT CTA | ATT AGA ACC CGC CTT TTC |
| AT | GT |
| AAA AGG CGG GTT CTA ATA | TGT ATT AGA ACC CGC CTT |
| CA | TT |
| AGG CGG GTT CTA ATA CAA | CTC TGC AGA ATT CCG GAC |
| T | AT |
| TTC TAA TAC AAT CTG CTG | AAC AGC AGA TTG TAT TAG |
| TT | AA |
| TAA TAC AAT CTG CTG TTG | GTC AAC AGC AGA TTG TAT |
| AC | TA |
| TAC AAT CTG CTG TTG ACG | CAC GTC AAC AGC AGA TTG |
| TG | TA |
| AAT CTG CTG TTG ACG TGA | ATT CAC GTC AAC AGC AGA |
| AT | TT |
| CGC TAA GGG AGG AGC CTA | GTA GGC TCC TCC CTT AGC |
| CG | GC |
| GCG GAC TAG AGT ATG GGA | GCTC CTC CCT TAG CGC GAG |
| G | |
| CTA GAG TAT GGG AGA GGG | CCA TAC TCT AGT CCG CCG |
| TG | GT |
| CCT CGC GCT AAG GGA GGA | TCT AGT CCG CCG GTT TCC |
| G | A |
| GGC GGA CTA GAG TAT GGG | GAC GTA GGC TCC TCC CTT |
| AG | AG |
| GGG AGG AGC CTA CGT CTG | TAC TCT AGT CCG CCG GTT |
| AT | T |
| CGC GCT AAG GGA GGA GCC | TCA GAC GTA GGC TCC TCC |
| T | CT |
| CGG ACC TCG CGC TAA GGG | CCT CCC TTA GCG CGA GGT |
| AG | CC |
| GGC GGA CTA GAG TAT GGG | TAG TGC GCC GGT TTC CAC |
| A | C |
| TAA GGG AGG AGC CTA CGT | ATT GTA TTA GAA CCC GCC |
| CT | T |

TABLE 9
Acidithiobacillus thiooxidans

| | |
|---|---|
| Sense primers | Antisense primers |

| | |
|---|---|
| GGG AGA CGA AAA GGT AAT | ATC CCC CGG TTT CTC CCT |
| CG | C |
| AAA GTT CTT TCG GTG ACG | ATA TTA GCG ATT ACC TTT |
| GG | T |
| CGG GGA AGG TTG ATA TGT | CAA CCT TCC CCG TCA CCG |
| TA | AA |
| GAG GGA GAA ACC GGG GGA | CCG AAG ATC CCC CGG TTT |
| T | CT |
| AAT CGC TAA TAT CGG TTA | CTC CAA TAG CAC GAG GTC |
| C | CG |
| CCG GGG GAT CTT CGG ACC | ACC GAT ATT AGC GAT TAC |
| TC | CT |
| TAA TAT CGCC TGC TGT TGA | AAG ATC CCC CGG TTT CTC |
| C | C |
| TCG GTG ACG GGG AAG GTT | TAT CAA CCT TCC CCG TCA |
| G | CC |
| GGA GAA ACC GGG GGA TCT | GGT TTC TCC CTC AGG ACG |
| T | TA |
| ACG TCC TGA GGG AGA AAC | GGT CCG AAG ATC CCC CGG |
| CG | TT |
| AGA CGA AAA GGT AAT CGC | TTT CAC GAC AGA CCT AAT |
| TA | G |
| GTG ACG GGG AAG GTT GAT | GTA ACC GAT ATT AGC GAT |
| A | TA |
| GAA ACC GGG GGA TCT TCG | ACA TAT CAA CCT TCC CCG |
| G | TC |
| TCC TGA GGG AGA AAC CGG | CCC GGT TTC TCC CTC AGG |
| GG | AC |
| CGA AAA GGT AAT CGC TAA | GCG ATT ACC TTT TCG TCT |
| TA | CC |
| AAA GGT AAT CGC TAA TAT | CCC CGT CAC CGA AAG AAC |
| CG | TT |
| TCG TGG GAG ACG AAA AGG | TTA ACA TAT CAA CCT TCC |
| TA | CC |
| CGG ACC TCG TGC TAT TGG | TTA GCG ATT ACC TTT TCG |
| AG | TC |
| GTT CTT TCG GTG ACG GGG | CTT CCC CGT CAC CGA AAG |
| A | AA |
| CTT TCG GTG ACG GGG AAG | ATT ACC TTT TCG TCT CCC |
| G | AC |

Archaea Kingdom:

TABLE 10
Acidianus sp.

| | |
|---|---|
| Sense primers | Antisense primers |

| | |
|---|---|
| GGG AAA CCG TGA GGG CGC | CCG CAT TGG GGA CGT TTC |
| T | GCG |
| GCG AAA CGT CCC CAA TGC | GCG CCC TCA CGG TTT CCC |
| GG | GCA |
| CCG CAG GGA AAC CGG TAA | CCG CAT TGG GGA CGT TTC |
| GCC | GCG |
| CCC GGG AAA GGG CAG TGA | GCG CCC TCA CGG TTT CCC |
| TA | GCA |
| GGG AAA GGG CAG TGA TAC | TTC CCG CAT TGG GGA CGT |
| T | TTC |
| AAT CCG GGG CAG GCG AAG | TAG CGC CCT CAC GGT TTC |
| GG | CC |
| AGG GTA CTG GAA CGT CCC | GGC TTA CCG GTT TCC CTG |
| TT | CG |
| AAG CGT CCG GCC AGA ACG | CTG CCC TTT CCC GGG TTG |
| CGC | A |
| CGC CTA AAG GGG CAT GGG | TCA CTG CCC TTT CCC GGG |
| CT | T |
| GGC TAT TTC CCG CTC ATG | GTA TCA CTG CCC TTT CCC |
| CC | G |
| CGT ACG CCC TCG GGT AAG | GCC CGG GTC TTT AAG CAG |
| AGG | TG |
| AAC GGC CCG CCA AAC CGA | CTC CCG CCC CCT AGC CCT |
| TA | GCA |
| AGC CGG CCC TGC AAG TCA | CCC GGG ATC TGT GGA TTT |
| C | CGC |
| CAC TGC TTA AAG ACC CGG | TAC CCG AGG GCG TAC GAC |
| G | T |
| GGA GCT AAT CCG GGG CAG | CCT CTT ACC CGA GGG CGT |
| GCG | ACG |
| AAA CCG TGA GGG CGC TAC | TTC GCC TGC CCC GGA TTA |
| CC | G |
| AGG CGA AGG GTA CTG GAA | GGC GGC AGG CTT ACC GGT |
| CGT | TTC |
| ACC CCC AGT GCT CCC GAA | CGG ATT AGC TCC AGT TTC |
| AG | CCG |
| CCC TTC GCC TAA AGG GGC | GGA CGT TCC AGT ACC CTT |
| ATG | C |
| GCA TGG GCT ATT TCC CGC | CCC CGG ATT AGC TCC AGT |
| TCA | TT |
| GGG AAA CCG TGA GGG CGC | TAC CCT TCG CCT GCC CCG |
| T | GAT |
| GCG AAA CGT CCC CAA TGC | CCA TGC CCC TTT AGG CGA |
| GG | A |

TABLE 11
Ferroplasma sp.

| | |
|---|---|
| Sense primers | Antisense primers |

| | |
|---|---|
| AGA GTC AAC CTG ACG AGC | AAG CTC GTC AGG TTG ACT |
| TTA | CT |
| GTC AAC CTG ACG AGC TTA | GTA AGC TCG TCA GGT TGA |
| CTC | C |
| TGA GAG TCA ACC TGA CGA | CGA GTA AGC TCG TCA GGT |
| GC | T |
| GAG CTT ACT CGA TAG CAG | CTG CTA TCG AGT AAG CTC |
| GAG | G |
| TTT AAT TCG AGA GGG TTA | TTT AAC CCT CTC GAA TTA |
| A | A |
| CTT ACT CGA TAG CAG GAG | CTC CTG CTA TCG AGT AAG |
| AGG | C |
| AAT CAA ATC TGA TGT CGG | TCA GAT TTG ATT TAA CCC |
| TGA | TC |
| GGT TAA ATC AAA TCT GAT | ACC CTC CTC ACC GAC ATC |
| G | AG |
| TTC GAG AGG GTT AAA TCA | ACA TCA GAT TTG ATT TAA |
| AAT | C |
| CAA ATC TGA TGT CGG TGA | CCG ACA TCA GAT TTG ATT |
| GGA | T |
| TAA ATC AAA TCT GAT GTC | TGA TTT AAC CCT CTC GAA |
| G | T |
| GAG AGG GTT AAA TCA AAT | TCA CCG ACA TCA GAT TTG |
| CTG | A |
| ATC TGA TGT CGG TGA GGA | ATT TGA TTT AAC CCT CTC |
| GGG | G |
| AAT TCG AGA GGG TTA AAT | CTA CCT GAT AGG TTG CAG |
| C | ACT |
| GAT GTC GGT GAG GAG GGT | GCA CCA CCT CTC TGC TAT |
| T | CG |
| GAG GGA TGG CAG TGT CGG | ATC CCT CAA CGG AAA AGC |
| A | A |
| TGG CCA AGA CTT TTC TCA | ACA CTT AAA GTG AAC GCC |
| T | CT |
| GAT GAG TCT GCA ACC TAT | TCG CTC CGA CAC TGC CAT |
| CA | C |
| TAG CAG AGA GGT GGT GCA | CCG ATC TCA TGT CTT GCA |
| TGG | GT |
| ACG GCC ACT GCT ATC AAG | ATG AGA AAA GTC TTG GCC |
| TTC | A |

TABLE 12
Metallosphaera sp.

| | |
|---|---|
| Sense primers | Antisense primers |

| | |
|---|---|
| AGG GCG TTA CCC CTA GTG | GGC ACT AGG GGT AAC GCC |
| C | C |
| TAC CCC TAG TGC CCT CGC | AGA AGC TCG ACC TCC CAC |
| A | CC |
| GCG CCC GTA GCC GGC CTG | TAC AGG CCG GCT ACG GGC |
| TAA | GC |
| GAG CTT CTC CTC CGC GAG | AGC TCG ACC TCC CAC CCC |
| GGG | G |
| GCA CCA GGC GCG GAA CGT | CCC CTC GCG GAG GAG AAG |
| CCC | C |
| GAG GTC GAG CTT CTC CTC | TGC GAG GGC ACT AGG GGT |
| CG | A |
| CCC TAG TGC CCT CGC AAG | TGA CTT TAC AGG CCG GCT |
| A | ACG |
| CCC GTA GCC GGC CTG TAA | CAT GGC TTA GCC CTA CCC |
| AGT | CTA |
| CGG GGT GGG AGG TCG AGC | AGG AGA AGC TCG ACC TCC |
| TTC | CA |
| GTC GAG CTT CTC CTC CGC | GAC GTT CCG CGC CTG GTG |
| GA | C |
| GGT GGG AGG TCG AGC TTC | CTT TAC AGG CCG GCT ACG |
| TCC | GG |
| TCG GGG TGG GAG GTC GAG | TCT TGC GAG GGC ACT AGG |
| C | G |
| GCG TTA CCC CTA GTG CCC | CGG AGG AGA AGC TCG ACC |
| T | TC |
| TAG GGG TAG GGC TAA GCC | TCG CGG AGG AGA AGC TCG |
| ATG | AC |
| CGC ACC AGG CGC GGA ACG | GAG GGC ACT AGG GGT AAC |
| T | G |
| GGG AGG TCG AGC TTC TCC | ACC CCG AGG GGC AAG AGG |
| T | CC |
| AGG TGG AGG AAT AAG CGG | GGG GTT ATC CAG ATC CCA |
| GG | AGG |
| GAA AGG TGG AGG AAT AAG | GCC ACG CCC TCT TCC CGA |
| C | GA |
| GGG AGT CGT ACG CTC TCG | GTT ATC CAG ATC CCA AGG |
| GGA | GC |
| CTA ACC TGC CCT TGG GAT | CTT ATT CCT CCA CCT TTC |
| CTG | TGG |

TABLE 13
Sulfolobus sp.

| | |
|---|---|
| Sense primers | Antisense primers |

| | |
|---|---|
| TAA ACC CTG CCG CAG TTG | CCA ACT GCG GCA GGG TTT |
| G | A |
| CCT TAA ACC CTG CCG CAG | ACT GCG GCA GGG TTT AAG |
| T | G |
| GTC CTG GAA CGG TTC CTC | CGA GGA ACC GTT CCA GGA |
| G | CTC |
| CTC TAC AAA GGC GGG GGA | AAC CGT TCC AGG ACT CCT |
| ATA | CG |
| CTG GAA CGG TTC CTC GCT | TCC AGG ACT CCT CGC CTA |
| GA | TGG |
| GGC GAG GAG TCC TGG AAC | CCT TTG TAG AGC GGG GAA |
| GGT | A |
| TTT CCC CGC TCT ACA AAG | AGC GAG GAA CCG TTC CAG |
| G | GA |
| TAC AAA GGC GGG GGA ATA | CGT TCC AGG ACT CCT CGC |
| AGC | CTA |
| CGC TCT ACA AAG GCG GGG | CCC CCG CCT TTG TAG AGC |
| G | G |
| ATA GGC GAG GAG TCC TGG | TTC AGC GAG GAA CCG TTC |
| AA | CA |
| CCA TAG GCG AGG AGT CCT | ATT CCC CCG CCT TTG TAG |
| G | A |
| GCT TTT CCC CGC TCT ACA | TTG TAG AGC GGG GAA AAG |
| A | C |
| GCT AAC CTA CCC TGA GGA | ATC TCC CTC CTC AGG GTA |
| GG | GGT |
| TCT CCC ATA GGC GAG GAG | GGG TTA TCT CCC TCC TCA |
| TC | G |
| TGG CTA ACC TAC CCT GAG | TCG CCT ATG GGA GAT TAT |
| G | C |
| ATA ATC TCC CAT AGG CGA | TCA GGG TAG GTT AGC CAC |
| G | GT |
| TGA GGA GGG AGA TAA CCC | CCT CAG GGT AGG TTA GCC |
| CG | A |
| ACA CGT GGC TAA CCT ACC | CCG GGG TTA TCT CCC TCC |
| CTG | T |
| CCT GAG GAG GGA GAT AAC | TCC TCG CCT ATG GGA GAT |
| C | T |
| AAA CTG GGG ATA ATC TCC | CCT CCT CAG GGT AGG TTA |
| C | G |

TABLE 14
Thermoplasma sp.

| | |
|---|---|
| Sense primers | Antisense primers |

| | |
|---|---|
| TCC TGA AAG GAC GAC CGG | CAG GGG CAT ATT CAC CGT |
| TG | AG |
| GGA CTG AGG GCT GTA ACT | TCA GGA TTA CAG GAT TTT |
| C | A |
| GAG GTT GAA TGT ACT TTC | ACC CTG AAA GTA CAT TCA |
| AGG | ACC |
| GGT GGC GAA AGC GTT CAA | GCC ACC GGT CGT CCT TTC |
| CT | A |
| GCC CTC ACG AAT GTG GAT | CTA GTT GAA CGC TTT CGC |
| T | C |
| ACC TCG AAA CCC GTT CGT | TCG TCC TTT CAG GAT TAC |
| AG | AGG |
| TCC GTA GTA ATC GTA GGT | ACG CTT TCG CCA CCG GTC |
| C | GTC |
| ATC CTG TAA TCC TGA AAG | GGG TTT CGA GGT TAG CTT |
| GAC | C |
| GTA GTC AGG ACT GAG GGC | CCC TCA GTC CTG ACT ACG |
| TG | A |
| AGG ACG ACC GGT GGC GAA | CTG AAG ATT TAT AAG ACC |
| AGC | GG |
| TAA CTC GCC CTC ACG AAT | TTA CAG CCC TCA GTC CTG |
| GT | ACT |
| GAA GGT GTT AAG TGG GTC | AAT CCA CAT TCG TGA GGG |
| A | CGA |
| AAA CCC GTT CGT AGT CAG | ATG GGG GTC TTG CTC GTT |
| GAC | AT |
| TAC GGT GAA TAT GCC CCT | GCT GTT GAC CTA CGA TTA |
| GC | C |
| CAC TTG GTG TTG CTT CTC | CCT ACG ATT ACT ACG GAA |
| CGT | TCC |
| GAT CAC TTT TAT TGA GTC | ACC CAC TTA ACA CCT TCG |
| T | C |
| AGC ATC AGG AAT AAG GGC | CCC AAG TCT TAC AGT CTC |
| TG | TT |
| AAG ACC CCC ATC TCT AAT | CTA CCC TGA AAG TAC ATT |
| T | CA |
| CCG GTC TTA TAA ATC TTC | CAG CCC TTA TTC CTG ATG |
| A | C |
| ATA ACG AGC AAG ACC CCC | GGT CGT CCT TTC AGG ATT |
| AT | AC |

In secondary PCRs a reaction is also included to quantify total bacteria or archaea present in the sample; in this case known universal primers are used for both kingdoms which are selected among the primers included in Table 15.

TABLE 15
Secondary PCR

Bacteria primers

| | |
|---|---|
| Eub27¹ F | AGA GTT TGA TCC TGG CTC AG |
| Univ533-F¹ | GTG CCA GCM GCC GCG GTA |
| Bact358-F² | CCT ACG GGA GGC AGC AG |
| Univ907-R³ | CCG TCA ATT CCT TTG AGT T |
| Bact338-R⁴ | GCT GCC TCC CGT AGG AGT |
| Bact1387-R⁵ | GGG CGG WGT GTA CAA GGC |
| Archaea primers | |
| Arch344-F⁶ | ACG GGG CGC AGC AGG CGC GA |
| Univ515-F⁷ | GTG CCA GCA GCC GCG GTA A |
| Arch958-R⁸ | YCC GGC GTT GAM TCC AAT T |
| Arch915-R⁴ | GTG CTC CCC CGC CAA TTC CT |
| Univ534-R⁵ | ATT ACC GCG GCT GCT GG |

¹Bond P., 2000, Appl Environ Microbiol. 66(9):3842-9.
²Schauer M, 2003, Aquat Microb Ecol Vol. 31: 163-174.
³Nakagawa T, 2002, FEMS Microbiology Ecology 41:199-209.
⁴Schrenk MO, 1998, Science. 279:1519-22.
⁵Ellis R, 2003, Appl Environ Microbiol. 69(6):3223-30.
⁶Casamayor EO, 2002, Environ Microbiol. 4(6):338-48.
⁷Edwards K, 2003, Appl Environ Microbiol. 69(5):2906-13.
⁸Orphan VJ, 2001, Appl Environ Microbiol. 66(2):700-11.

Each secondary PCR has a specific cycle, wherein the alignment temperature changes, said temperature being specific for each used primer pair. Table 16 summarizes general conditions for all qPCR cycles.

TABLE 16

| | Step | Temperature (° C.) | Time (s) |
|---|---|---|---|
| 1 | Initial denaturation | 95 | 120 |
| 2 | Denaturation | 95 | 30 |
| 3 | Alignment | (*) | 30 |
| 4 | Extension | 72 | 40 |
| 5 | Pre-reading | 80 | 10 |
| 6 | Reading | 80 | — |

| | Repeat 40 times from step 2 to step 6 (qPCR cycle) | |
|---|---|---|
| 7 | Denaturation curve | Between 70 and 100° C., reading each |
| | | 0.2° C. |

(*) specific temperature for each used primer pair

Duration curve carried out at the end of cycle 40, gives the Tm of the amplification product, and is also used to establish whether more than one amplification product is present in the amplified sample, as each would generate its own curve.

The PCR thermocycler gives a result corresponding to DNA concentration present in each reaction, and this information is used to calculate the number of microorganisms present in the sample, which is called Q. This value is inferred by the computational program associated to the thermocycler based on: DNA concentration in calibration curve reactions and the cycle in which sample begins to amplify (or to exponentially increase its fluorescence value). The correlation between the logarithm of DNA concentration and the cycle in which amplification is observed generates a linear equation, from which DNA concentration in the analyzed samples is inferred.

Calculation of the Number of Microorganisms Present in the Sample

Taking into account the qPCR result and other data generated during the process, the inventors have developed a mathematical formula that allows calculating the exact number of microorganisms from a given taxon present in a given sample, specially a biomining sample. The formula is as follows:

$$Mo / Um = \frac{Q \times T}{5 \cdot {10}^{- 6} [ ng \text{/} mo ] \times U \times Cm}$$

where:
Mo/Um is the number of microorganisms, either bacteria or archaea, per sample unit;
Q is the amount of initial DNA in nanograms that is present in each secondary PCR reaction, as determined by the program associated to the qPCR equipment;
T is the amount of total DNA extracted from the sample;
U is the amount of DNA used in the primary PCR reaction; and
Cₘ is the amount of biomining sample from which DNA was extracted, expressed in ml for liquid samples or in g for solid samples.
The number 5×10⁻⁶ ng/mo is the average amount of DNA nanograms contained in the genome of a microorganism, according to Kuske et al. (1998).

By applying the method of the invention, the number of microorganisms belonging to the taxons Acidiphilium sp., Leptospirillum sp., Sulfobacillus sp. Acidithiobacillus ferrooxidans and Acidithiobacillus thiooxidans, Acidianus sp., Ferroplasma sp, Sulfolobus sp., Metallosphaera sp, and/or Thermoplasma sp. present in a sample can be determined.

### EXAMPLES

### Example 1

### Quantification of Acidithiobacillus thiooxidans, Acidithiobacillus ferrooxidans, Leptospirillum sp. and Acidiphilium sp. Present in a Sample

Five solid samples obtained from mineral bioleaching heaps (MS-1 to MS-5) and 2 liquid samples recovered from bioleaching effluents (ML-1 and ML-2) were analyzed and total DNA was extracted from each one.

For all solid samples a further step was necessary, a re-purification of DNA, which consisted in a sample re-purification using any existing purification technique; in our laboratories this step is performed using commercial DNA purification columns to obtain a translucent appearance in the extraction solution.

Then, total DNA was quantified in each sample using a NanoDrop 1.0 spectrophotometer. Total extracted DNA nanograms (T) are shown in Table 17 together with the initial sample volumes (Cₘ). Registered results were:

TABLE 17

| Sample | T | Cₘ | |
|---|---|---|---|
| MS-1 | 316.8 | 0.5 | g |
| MS-2 | 370.4 | 0.5 | g |
| MS-3 | 315.2 | 0.5 | g |
| MS-4 | 526.4 | 0.5 | g |
| MS-5 | 400 | 5.0 | g |
| ML-1 | 2938 | 81.00 | ml |
| ML-2 | 1114 | 76.55 | ml |

Each of these samples was diluted with sterile nuclease-free water in order to obtain a concentration between 0.5 and 30 ng/μl. Table 18 shows the final volume to which the DNA solution was brought and its final concentration.

TABLE 18

| Sample | Final volume (μl) | Concentration (ng/μl) |
|---|---|---|
| MS-1 | 80 | 3.96 |
| MS-2 | 80 | 4.63 |
| MS-3 | 80 | 3.94 |
| MS-4 | 80 | 6.58 |
| MS-5 | 80 | 5.00 |
| ML-1 | 100 | 29.38 |
| ML-2 | 100 | 11.14 |

A calibration curve was simultaneously prepared to allow the calculation of DNA concentration in experimental samples. Four serial dilutions were prepared from a standard DNA mix containing 25 ng of DNA from each of the following microorganisms: Acidithiobacillus thiooxidans, Acidithiobacillus ferrooxidans, Leptospirillum sp. and Acidiphilium sp., in a final volume of 30 μl, to obtain 100 ng of total DNA in the standard sample, which in its turn is part of the calibration curve.

More specifically, DNA was used from the following strains:
A. ferrooxidans DSM 16786;
A. thiooxidans DSM 504;
Leptospirillum sp. DSM 1931 and
Acidiphilium acidophilus DSMZ 700.

The reaction mix for the primary PCR was prepared, wherein the amount of each constituent was multiplied by the total number of reactions to be carried out; a single reaction mix was prepared in order to homogenize reagent concentrations in the different PCR tubes. The reaction mix was aliquoted in 0.2 ml tubes, using a volume of 24 μl of reaction mix per tube.

In the present Example, the following reactions were performed in duplicate:
a) seven reactions for the samples and
b) 5 reactions for the calibration curve, corresponding to standard DNA master mix concentrations of 1×, 0.1×, 0.01×, 0.001× and 0.0001×, and a blank, giving a total of 25 reactions.

The prepared mix is shown in Table 19.

TABLE 19

| Reagent | 1 reaction | 25 reactions |
|---|---|---|
| Sterile nuclease-free H₂O | 18.35 μl | 458.75 μl |
| PCR Buffer 10x | 2.5 μl | 62.5 μl |
| MgCl₂ (50 mM) | 1.5 μl | 37.5 μl |
| dNTPs (10 mM each) | 0.5 μl | 12.5 μl |
| Primer Bacteria 27F (10 μM) | 0.5 μl | 12.5 μl |
| Primer Bacteria 1492R (10 μM) | 0.5 μl | 12.5 μl |
| Hot Start Taq (5 U/μl) | 0.15 μl | 3.75 μl |

Used primers are described in Table 20.

TABLE 20

| Microorganism | Alignment | |
|---|---|---|
| to be | tempera- | |
| determined | ture | Used primers |
| Total bacteria | 59° C. | Eubac27F: |
| | | AGA GTT TGA TCC TGG CTC AG |
| | | Univ1492R: |
| | | GGT TAC CTT GTT ACG ACT T |

This primary PCR reaction mix was homogenized and 25 aliquots were made with 24 μl each in 0.2 ml tubes appropriately labeled. To this mix 1 μl of sample DNA dilutions or 1 μl of calibration curve DNA was added as appropriate. To the primary PCR negative control 1 μl of sterile nuclease-free water was added instead of DNA.

Reactions were incubated in a MJ Research PTC-100 thermocycler, with the following cycle program:

TABLE 21

| | Temperature | |
|---|---|---|
| Step | (° C.) | Time (s) |
| 1. Initial denaturation | 95 | 120 |
| 2. Denaturation | 95 | 30 |
| 3. Alignment | 56 | 30 |
| 4. Extension | 72 | 120 |

Wherein steps 2 to 4 were repeated 18 times.

Subsequently 5 secondary PCR were performed, one for each taxon: Acidithiobacillus thiooxidans, Acidithiobacillus ferrooxidans, Leptospirillum sp., Acidiphilium sp. and one for total bacteria, using specific primers for each of them, which hybridize inside the region amplified in the primary PCR. Sense and antisense primers were selected for the different taxons from those included in the description of the tables corresponding to each taxon, Tables 5, 6, 8 and 9 in this case. On the other hand, for total bacteria primers described in the literature were used, which were included in Table 15.

Primers used for each taxon and their respective annealing temperatures are indicated in Table 22.

TABLE 22

| Microorganism | Alignment | |
|---|---|---|
| to be | tempera- | |
| determined | ture | Used primers |
| Total bacteria | 56° C. | (P.1) 533-F: |
| | | 5′- GTG CCA GCA GCC GCG |
| | | GTA -3′ |
| | | (P.2) 907-R: |
| | | 5′- CCG TCA ATT CCT TTG |
| | | AGT T -3′ |
| A. ferrooxidans | 60° C. | (P.1) F: |
| | | 5′- GTG GAG GAC GAA AAG |
| | | GCG G -3′ |
| | | (P.2) R: |
| | | 5′- ATT AGA ACC CGC CTT |
| | | TTC GT -3′ |
| A. thiooxidans | 56° C. | (P.1) F: |
| | | 5′- AAA GGT AAT CGC TAA |
| | | TAT CG -3′ |
| | | (P.2) R: |
| | | 5′- ATT ACC TTT TCG TCT |
| | | CCC AC -3′ |
| Leptospirillum | 58° C. | (P.1) F: |
| sp. | | 5′- AAC AAG GTA CCC GTC |
| | | TAG A -3′ |
| | | (P.2) R: |
| | | 5′- CTA GAC GGG TAC CTT |
| | | GTT AC -3′ |
| Acidiphilium | 61° C. | (P.1) F: |
| sp. | | 5′- AGG AGG CAG TCA ACC |
| | | ACG GT -3′ |
| | | (P.2) R: |
| | | 5′- GTT AGC GCA TCA ACT |
| | | TAA GG -3′ |

One qPCR was carried out on each primary PCR reaction product for each taxon to be determined, these reactions being performed in duplicate. The qPCR was carried out using Mix SYBR Green qPCR. For each secondary PCR one duplicate per each one of the 25 primary PCR reactions is considered plus one control, which gives a total of 51 reactions. For each PCR the reaction mix shown in Table 23 is prepared, where primers are those that are corresponding according to Table 22.

TABLE 23

| | 1 reaction | 51 reactions |
|---|---|---|
| Sterile nuclease-free H₂O | 16.1 μl | 821.1 μl |
| Primer 1 (10 μM) | 0.5 μl | 25.5 μl |
| Primer 2 (10 μM) | 0.5 μl | 25.5 μl |
| PCR Buffer 10x | 2.5 μl | 127.5 μl |
| MgCl₂ (50 mM) | 1.5 μl | 76.5 μl |
| dNTPs (10 mM each) | 2.5 μl | 127.5 μl |
| Hot Start Taq (5 U/μl) | 0.15 μl | 7.65 μl |
| SYBR Green qPCR 100x | 0.25 μl | 12.75 μl |

This reaction mix was homogenized and aliquoted in 51 0.2 ml tubes, which were duly labeled. To each of the tubes 1 μl of primary PCR or 1 μl of sterile nuclease-free water for the blank was added.

PCR tubes containing the reaction mix and sample were vortexed for 5 seconds and centrifuged for 1 minute at 2000 rpm, in order to homogenize and bring the reaction liquid to the bottom of the tube, respectively.

Then, the tubes with secondary PCR reactions were subjected to temperature cycles for amplification. According to the microorganism to be determined, different primer pairs were used and therefore different amplification programs were used. In the following Table, amplification programs used in the different secondary PCR reactions are shown.

TABLE 24

| | Step | Temperature (° C.) | Time (s) |
|---|---|---|---|
| 1 | Initial denaturation | 95 | 120 |
| 2 | Denaturation | 95 | 30 |
| 3 | Alignment | (*) | 30 |
| 4 | Extension | 72 | 40 |
| 5 | Pre-reading | 80 | 10 |
| 6 | Reading | 80 | — |

| | Repeat 40 times from step 2 to step 6 (qPCR cycle) | |
|---|---|---|
| 7 | Denaturation curve | Between 70 and 100° C., reading each |
| | | 0.2° C. |

(*) specific temperature for each used primer pair, as indicated in Table 22.

When the qPCR is finished, all data generated by the qPCR thermocycler are stored; this data corresponds to Q and is shown in Table 25, wherein DNA amounts in nanograms used for each reaction are included (U).

TABLE 25

| | Q | | | | | |
|---|---|---|---|---|---|---|
| Sample | Total bacteria | A. ferrooxidans | A. thiooxidans | Leptospirillum sp. | Acidiphilium sp. | U |
| MS-1 | 187.68 × 10⁻³ | 1.63 × 10⁻³ | 0.13 × 10⁻³ | 25.36 × 10⁻³ | 0.03 × 10⁻³ | 2 |
| MS-2 | 33.91 × 10⁻³ | 0.51 × 10⁻³ | 2.33 × 10⁻³ | 1.63 × 10⁻³ | 0 | 2 |
| MS-3 | 149.60 × 10⁻³ | 0.59 × 10⁻³ | 0.03 × 10⁻³ | 10.46 × 10⁻³ | 0.008 × 10⁻³ | 2 |
| MS-4 | 71.08 × 10⁻³ | 0.01 × 10⁻³ | 0.03 × 10⁻³ | 6.23 × 10⁻³ | 0.002 × 10⁻³ | 2 |
| MS-5 | 142.68 × 10⁻³ | 0.29 × 10⁻³ | 3.20 × 10⁻³ | 15.10 × 10⁻³ | 0 | 2 |
| ML-1 | 9.20 × 10⁻³ | 0 | 0 | 0.26 × 10⁻³ | 0 | 2 |
| ML-2 | 49.03 × 10⁻³ | 0.05 × 10⁻³ | 0 | 2.15 × 10⁻³ | 0 | 2 |

### Calculation of the Number of Microorganisms Present in the Samples

Taking into account the qPCR result and other data generated during the process, the following formula was applied, the meaning of which was defined above:

$$Mo / Um = \frac{Q \times T}{5 \cdot {10}^{- 6} [ ng \text{/} mo ] \times U \times Cm}$$

According to this, the following microbiological populations were determined in the analyzed samples:

TABLE 26
MS-1

| Bacteria | Mo./g of sample |
|---|---|
| Total bacteria | 1.19 × 10⁷ |
| A. ferrooxidans | 1.03 × 10⁵ |
| A. thiooxidans | 8.03 × 10³ |
| Leptospirillum sp. | 1.61 × 10⁶ |
| Acidiphilium sp. | 2.11 × 10³ |

TABLE 27
MS-2

| Bacteria | Mo./g of sample |
|---|---|
| Total bacteria | 2.51 × 10⁶ |
| A. ferrooxidans | 3.81 × 10⁴ |
| A. thiooxidans | 1.72 × 10⁵ |
| Leptospirillum sp. | 1.21 × 10⁵ |
| Acidiphilium sp. | 0 |

TABLE 28
MS-3

| Bacteria | Mo./g of sample |
|---|---|
| Total bacteria | 9.43 × 10⁶ |
| A. ferrooxidans | 3.70 × 10⁴ |
| A. thiooxidans | 2.11 × 10³ |
| Leptospirillum sp. | 6.60 × 10⁵ |
| Acidiphilium sp. | 5.07 × 10² |

TABLE 29
MS-4

| Bacteria | Mo./g of sample |
|---|---|
| Total bacteria | 7.48 × 10⁶ |
| A. ferrooxidans | 1.03 × 10³ |
| A. thiooxidans | 2.78 × 10³ |
| Leptospirillum sp. | 6.56 × 10⁵ |
| Acidiphilium sp. | 2.45 × 10² |

TABLE 30
MS-5

| Bacteria | Mo./g of sample |
|---|---|
| Total bacteria | 1.14 × 10⁶ |
| A. ferrooxidans | 3.31 × 10³ |
| A. thiooxidans | 2.56 × 10⁴ |
| Leptospirillum sp. | 1.21 × 10⁵ |
| Acidiphilium sp. | 0 |

TABLE 31
ML-1

| Bacteria | Mo./ml of sample |
|---|---|
| Total bacteria | 3.34 × 10⁴ |
| A. ferrooxidans | 0 × 10⁰ |
| A. thiooxidans | 0 × 10⁰ |
| Leptospirillum sp. | 9.47 × 10² |
| Acidiphilium sp. | 0 |

TABLE 32
ML-2

| Bacteria | Mo./ml of sample |
|---|---|
| Total bacteria | 7.13 × 10⁴ |
| A. ferrooxidans | 6.69 × 10¹ |
| A. thiooxidans | 0 × 10⁰ |
| Leptospirillum sp. | 3.12 × 10³ |
| Acidiphilium sp. | 0 |

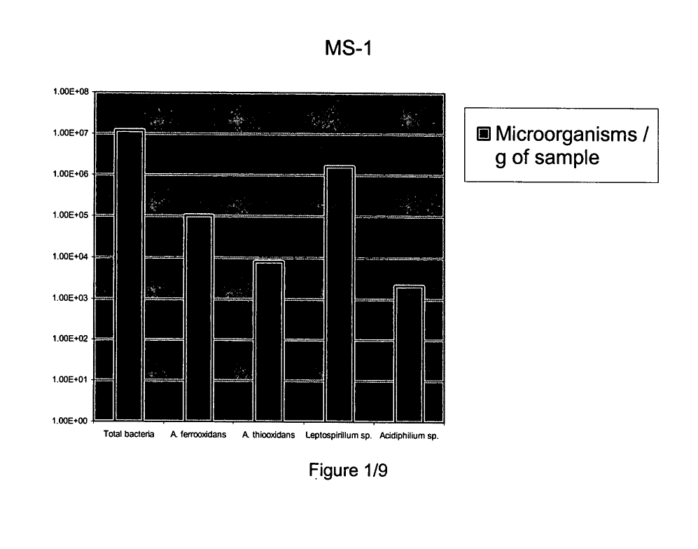

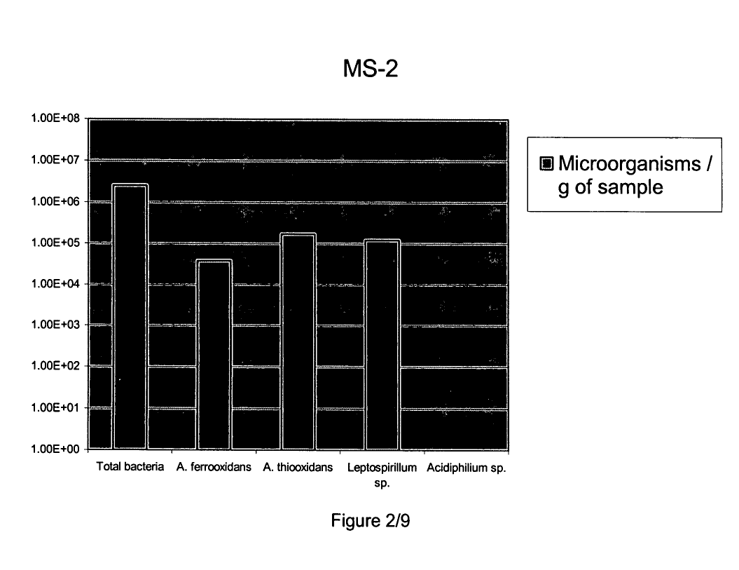

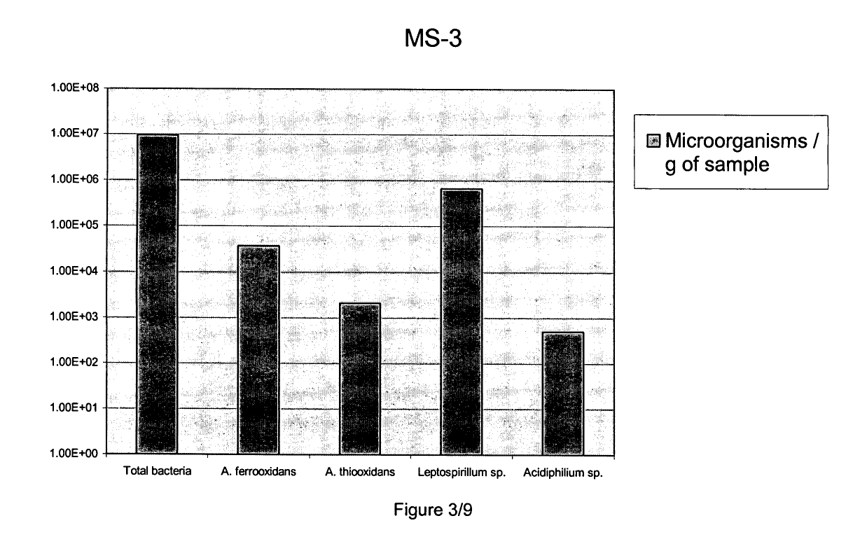

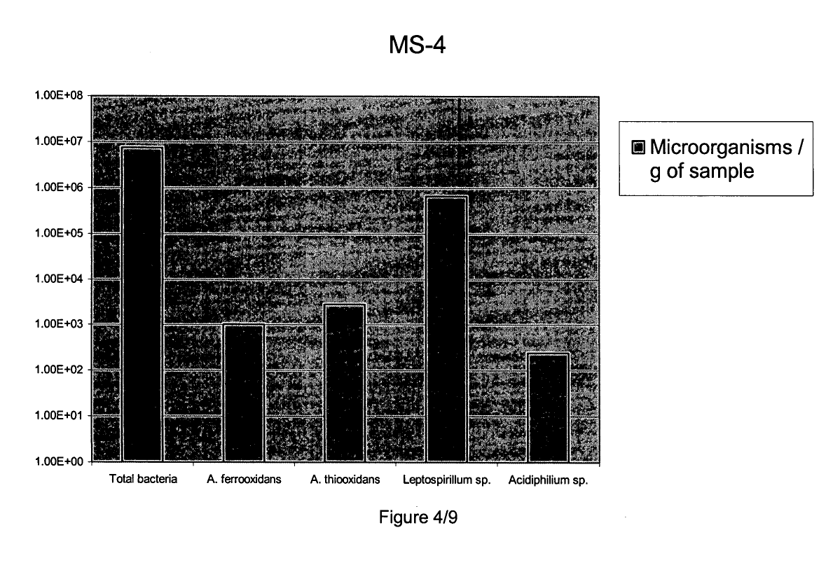

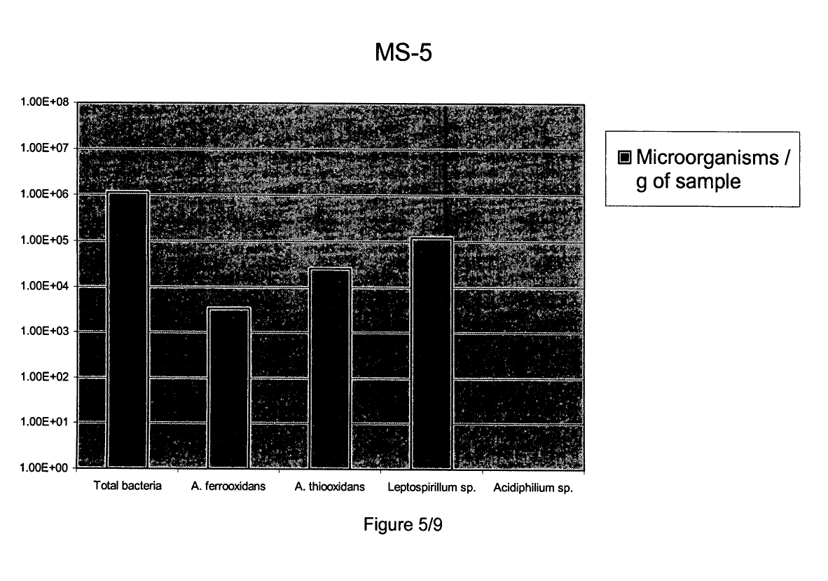

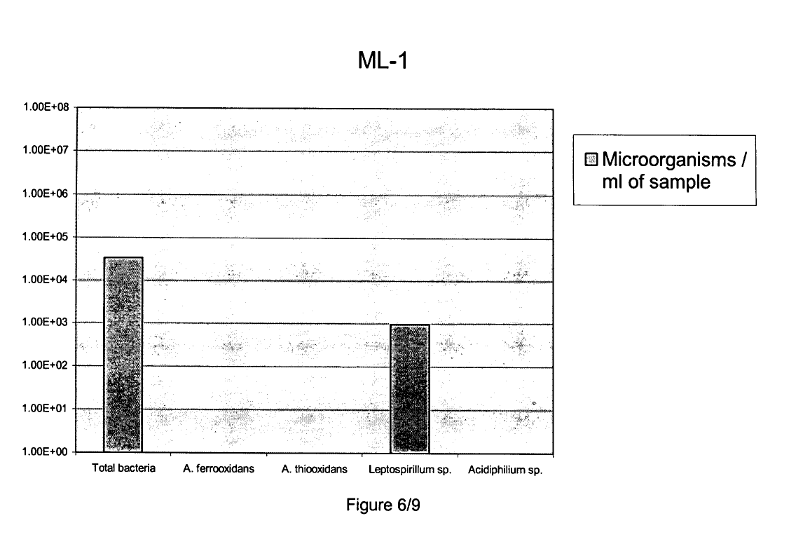

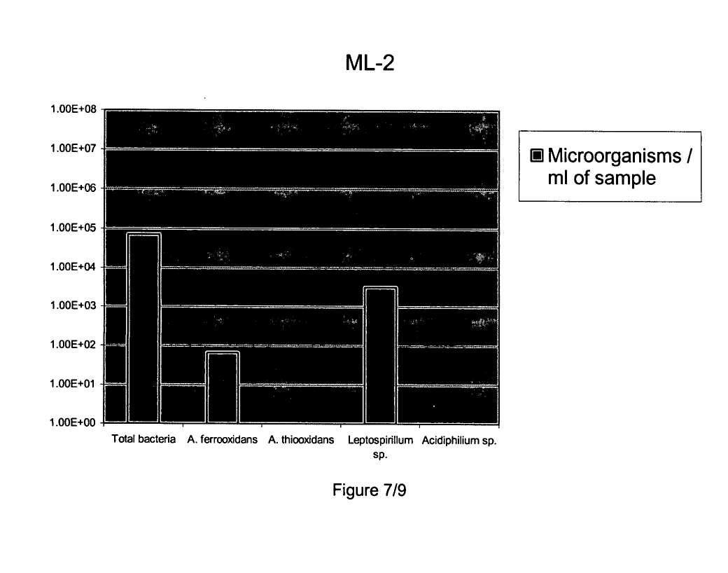

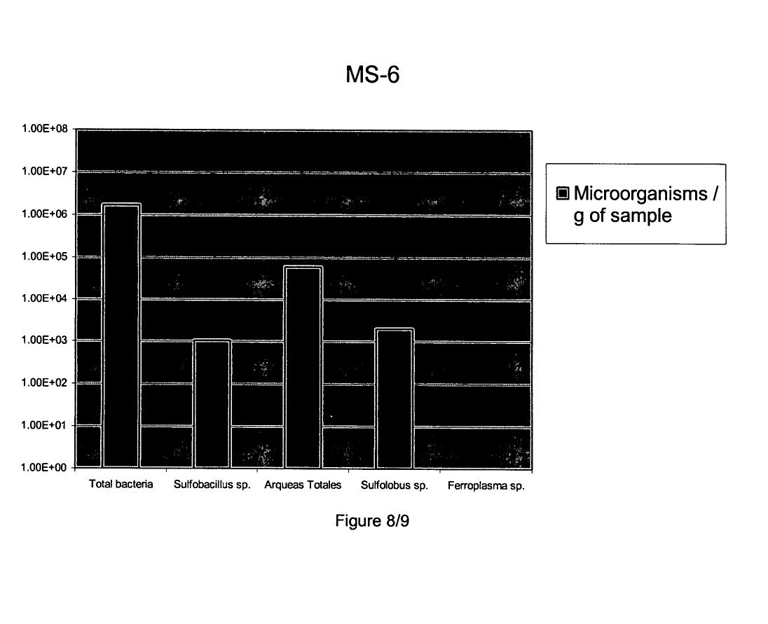

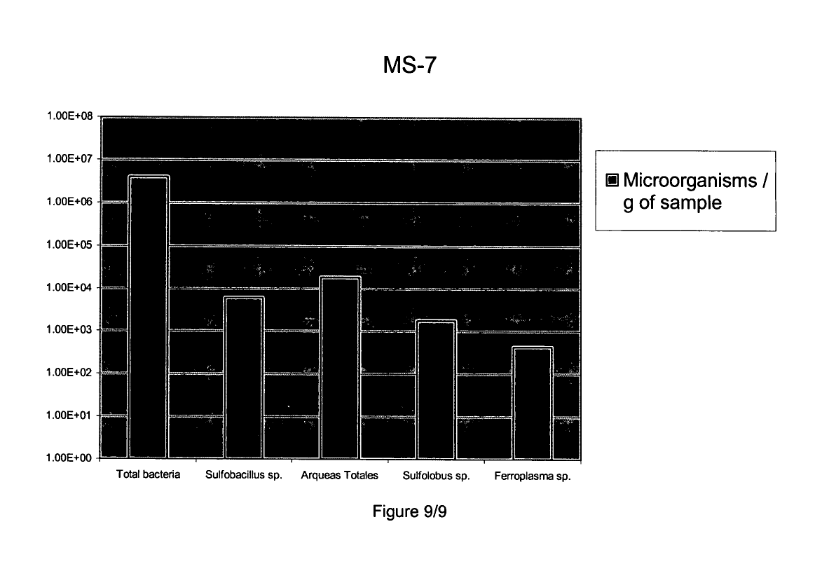

FIGS. 1 to 7 are plots of the results described in Tables 26 to 32.

### Example 2

### Quantification of Sulfobacillus sp., Sulfolobus sp., and Ferroplasma sp. in a Sample

Two solid samples obtained from mineral bioleaching heaps (MS-6 and MS-7) were analyzed and total DNA was extracted from each one.

A further DNA re-purification step was required to obtain a translucent appearance in the extraction solution.

Then, total DNA was quantified in each sample using a NanoDrop 1.0 spectrophotometer. Total extracted DNA nanograms (T) are shown in Table 34 together with the initial sample volumes (Cₘ). Results are shown in Table 33.

TABLE 33

| Sample | T | Cₘ |
|---|---|---|
| MS-6 | 426.8 | 0.5 g |
| MS-7 | 277.2 | 0.5 g |

Each of these samples was diluted with sterile nuclease-free water in order to obtain a concentration between 0.5 and 30 ng/μl. Table 34 shows the final volume to which the DNA solution was brought and its final concentration.

TABLE 34

| Sample | Final volume (μl) | Concentration (ng/μl) |
|---|---|---|
| MS-1 | 80 | 5.34 |
| MS-2 | 80 | 3.47 |

Two calibration curves were prepared simultaneously, one for the Bacteria kingdom and another for the Archaea kingdom, which allowed calculating DNA concentration in experimental samples. For the Bacteria kingdom 4 serial dilutions were carried out from a DNA standard, hereinafter called Bacteria standard, containing 100 ng of Sulfobacillus sp. DNA in a final volume of 30 μl, being the standard solution also part of the calibration curve.

More specifically, DNA was used from the strain:
Sulfobacillus sp. DSM 10 332

For the Archaea kingdom, four serial dilutions were prepared from a standard DNA mix containing 50 ng of DNA from each of the following microorganisms: Sulfolobus sp. and Ferroplasma sp. in a final volume of 30 μl, obtaining 100 ng of total DNA in the standard mix, hereinafter called Archaea standard, which is also part of the calibration curve.

More specifically, DNA was used from the following strains:
Sulfolobus sp. DSM 6482 and
Ferroplasma sp. DSM 12658.

Then, reaction mixes for the primary PCR were prepared, wherein the amount of each constituent was multiplied by the total number of reactions to be carried out; a single reaction mix was prepared in order to homogenize reagent concentrations in the different PCR tubes. The reaction mix was aliquoted in 0.2 ml tubes, using a volume of 24 μl of reaction mix per tube.

For the Bacteria kingdom and Sulfobacillus determination, the following reactions were set up in duplicate:
a) two reactions for the samples and
b) 5 reactions for the calibration curve, corresponding to the Bacteria standard solution in concentrations of 1×, 0.1×, 0.01×, 0.001× and 0.0001×, and a blank, giving a total of 15 reactions.

The prepared mix is shown in Table 35.

TABLE 35

| Reagent | 1 reaction | 15 reactions | |
|---|---|---|---|
| Sterile nuclease-free H₂O | 18.35 μl | 275.25 | μl |
| PCR Buffer 10× | 2.5 μl | 37.5 | μl |
| MgCl₂ (50 mM) | 1.5 μl | 22.5 | μl |
| dNTPs (10 mM each) | 0.5 μl | 7.5 | μl |
| Primer Bacteria 27F (10 μM) | 0.5 μl | 7.5 | μl |
| Primer Bacteria 1492R (10 μM) | 0.5 μl | 7.5 | μl |
| Hot Start Taq (5 U/μl) | 0.15 μl | 2.25 | μl |

Primers were those described in Table 36:

TABLE 36

| Microorganism | Alignment | |
|---|---|---|
| to be | tempera- | |
| determined | ture | Used primers |
| Total bacteria | 59° C. | Eubac27F: |
| | | AGA GTT TGA TCC TGG CTC AG |
| | | Univ1492R: |
| | | GGT TAC CTT GTT ACG ACT T |

This primary PCR reaction mix was homogenized and 15 aliquots were made with 24 μl each in 0.2 ml tubes appropriately labeled. To this mix 1 μl of sample DNA dilutions or 1 μl of calibration curve DNA was added as appropriate. To the negative control 1 μl of sterile nuclease-free water was added instead of DNA.

Reactions were incubated in a MJ Research PTC-100 thermocycler, with the following cycle program:

TABLE 37

| | Temperature | |
|---|---|---|
| Step | (° C.) | Time (s) |
| 1. Initial denaturation | 95 | 120 |
| 2. Denaturation | 95 | 30 |
| 3. Alignment | 62 | 30 |
| 4. Extension | 72 | 120 |

Wherein steps 2 to 4 were repeated 18 times.

For the Archaea kingdom and Sulfolobus sp. and Ferroplasma sp. determination, the following reactions were set up in duplicate:
a) two reactions for the samples and
b) 5 reactions for the calibration curve, corresponding to the Archaea standard solution in concentrations of 1×, 0.1×, 0.01×, 0.001× and 0.0001 ×, and a blank, giving a total of 15 reactions.

The prepared mix is shown in Table 38.

TABLE 38

| Reagent | 1 reaction | 15 reactions |
|---|---|---|
| Sterile nuclease-free H₂O | 18.35 μl | 275.25 μl |
| PCR Buffer 10x | 2.5 μl | 37.5 μl |
| MgCl₂ (50 mM) | 1.5 μl | 22.5 μl |
| dNTPs (10 mM each) | 0.5 μl | 7.5 μl |
| Primer Archaea 21F (10 μM) | 0.5 μl | 7.5 μl |
| Primer Archaea 1492R (10 μM) | 0.5 μl | 7.5 μl |
| Hot Start Taq (5 U/μl) | 0.15 μl | 2.25 μl |

Primers were those described in Table 39:

TABLE 39

| Microorganism | Alignment | |
|---|---|---|
| to be | tempera- | |
| determined | ture | Used primers |
| Total archaea | 57° C. | Arch21F: |
| | | TTC CGG TTG ATC CTG CCG GA |
| | | Univ1492R: |
| | | GGT TAC CTT GTT ACG ACT T |

This primary PCR reaction mix was homogenized and 15 aliquots were made with 24 μl each in 0.2 ml tubes appropriately labeled. To this mix 1 μl l of sample DNA dilutions or 1 μl of calibration curve DNA was added as appropriate. To the negative control 1 μl of sterile nuclease-free water was added instead of DNA.

Reactions were incubated in a MJ Research PTC-100 thermocycler, with the following cycle program:

TABLE 40

| | Temperature | |
|---|---|---|
| Step | (° C.) | Time (s) |
| 1. Initial denaturation | 95 | 120 |
| 2. Denaturation | 95 | 30 |
| 3. Alignment | 57 | 30 |
| 4. Extension | 72 | 120 |

Wherein steps 2 to 4 were repeated 18 times.

Subsequently, 5 secondary PCR were performed, two on the primary PCR reaction product for the Bacteria kingdom, for Sulfobacillus sp. and for total bacteria; and three on the primary PCR reaction product for the Archaea kingdom, for Sulfolobus sp. and Ferroplasma sp. and for total archaea, using specific primers for each of them that hybridize inside the region amplified in the primary PCR. Sense and antisense primers were selected for the different genera from those included in the description of the tables corresponding to each taxon, Tables 7, 11 and 13 in this case. On the other hand, for total bacteria or archaea primers described in the literature were used, which were included in Table 15.

Primers used for each taxon and their respective annealing temperatures are indicated in Table 41.

TABLE 41

| Microorganism | Alignment | |
|---|---|---|
| to be | tempera- | |
| determined | ture | Used primers |
| Total bacteria | 59 | (P.1) 27-F: |
| | | 5′- AGA GTT TGA TCC TGG |
| | | CTC AG -3′ |
| | | (P.2) 338-R: |
| | | 5′- GCT GCC TCC CGT AGG |
| | | AGT -3′ |
| Sulfobacillus | 66 | (P.1) F: |
| sp. | | 5′- AGG TGT CGC GGG GGT |
| | | CCA CC -3′ |
| | | (P.2) R: |
| | | 5′- CCA GGA ATT CCA TGC |
| | | ACC TC -3′ |
| Total archaea | 60 | (P.1) 515-F: |
| | | 5′- GTG CCA GCA GCC GCG |
| | | GTA A -3′ |
| | | (P.2) 958-R: |
| | | 5′- TCC GGC GTT GAA TCC |
| | | AAT T -3′ |
| Sulfolobus sp. | 60 | (P.1) F: |
| | | 5′- TAA ACC CTG CCG CAG |
| | | TTG G -3′ |
| | | (P.2) R: |
| | | 5′- CCA ACT GCG GCA GGG |
| | | TTT A -3′ |
| Ferroplasma sp. | 56 | (P.1) F: |
| | | 5′- GAT GTC GGT GAG GAG |
| | | GGT T -3′ |
| | | (P.2) R: |
| | | 5′- ATT TGA TTT AAC CCT |
| | | CTC G -3′ |

One qPCR was carried out on each primary PCR reaction product for each taxon to be determined, these reactions being performed in duplicate. The qPCR was carried out using Mix SYBR Green qPCR. For each secondary PCR one duplicate per each one of the 15 primary PCR reactions is considered plus one control, which gave a total of 31 reactions. For each PCR the reaction mix shown in Table 42 was prepared, where primers are those that are corresponding according to Table 41.

TABLE 42

| | 1 reaction | 31 reactions |
|---|---|---|
| Sterile nuclease-free H₂O | 16.1 μl | 499.1 μl |
| Primer 1 (10 μM) | 0.5 μl | 15.5 μl |
| Primer 2 (10 μM) | 0.5 μl | 15.5 μl |
| PCR Buffer 10x | 2.5 μl | 77.5 μl |
| MgCl₂ (50 mM) | 1.5 μl | 46.5 μl |
| dNTPs (10 mM each) | 2.5 μl | 77.5 μl |
| Hot Start Taq (5 U/μl) | 0.15 μl | 4.65 μl |
| SYBR Green qPCR 100x | 0.25 μl | 7.75 μl |

This reaction mix was homogenized and aliquoted in 31 0.2 ml tubes, which were duly labeled. To each of the tubes 1 μl of primary PCR or 1 μl of sterile nuclease-free water for the blank was added.

PCR tubes containing the reaction mix and sample were vortexed for 5 seconds and centrifuged for 1 minute at 2000 rpm, in order to homogenize and bring the reaction liquid to the bottom of the tube, respectively.

Then, the tubes with secondary PCR reactions were subjected to temperature cycles for amplification. According to the microorganism to be determined, different primer pairs were used and therefore different amplification programs were used. In the following Table, amplification programs used in the different secondary PCR reactions are shown.

TABLE 43

| | Step | Temperature (° C.) | Time (s) |
|---|---|---|---|
| 1 | Initial denaturation | 95 | 120 |
| 2 | Denaturation | 95 | 30 |
| 3 | Alignment | (*) | 30 |
| 4 | Extension | 72 | 40 |
| 5 | Pre-reading | 80 | 10 |
| 6 | Reading | 80 | — |

| | Repeat 40 times from step 2 to step 6 (qPCR cycle) | |
|---|---|---|
| 7 | Denaturation curve | Between 70 and 100° C., |
| | | reading each 0.2° C. |

(*) specific temperature for each used primer pair, as indicated in Table 41.

When the qPCR is finished, all data generated by the qPCR thermocycler are stored; this data corresponds to Q and is shown in Table 44, wherein DNA amounts in nanograms used for each reaction are included (U).

TABLE 44

| | Q | | | | | |
|---|---|---|---|---|---|---|
| Sample | Total bacteria | Sulfobacillus sp. | Total archaea | Sulfolobus sp. | Ferroplasma sp | U |
| MS-6 | 20.26 × 10⁻³ | 0.01 × 10⁻³ | 0.71 × 10⁻³ | 0.05 × 10⁻³ | 0 | 2 |
| MS-7 | 72.51 × 10⁻³ | 0.11 × 10⁻³ | 0.34 × 10⁻³ | 0.03 × 10⁻³ | 0.007 × 10⁻³ | 2 |

### Calculation of the Number of Microorganisms Present in the Sample

Taking into account the qPCR result and other data generated during the process, the following formula was applied, the meaning of which was defined above:

$$Mo / Um = \frac{Q \times T}{5 \cdot {10}^{- 6} [ ng \text{/} mo ] \times U \times Cm}$$

According to this, the following microbiological populations were determined in the analyzed samples:

TABLE 45
MS-6

| Microorganism | Mo./g of sample |
|---|---|
| Total bacteria | 1.73 × 10⁶ |
| Sulfobacillus sp. | 1.05 × 10³ |
| Total archaea | 6.04 × 10⁴ |
| Sulfolobus sp. | 2.02 × 10³ |
| Ferroplasma sp. | 0 |

TABLE 46
MS-7

| Microorganism | Mo./g of sample |
|---|---|
| Total bacteria | 4.02 × 10⁶ |
| Sulfobacillus sp. | 6.00 × 10³ |
| Total archaea | 1.89 × 10⁴ |
| Sulfolobus sp. | 1.76 × 10³ |
| Ferroplasma sp. | 4.33 × 10² |

FIGS. 8 and 9 are plots of the results described in Tables 45 and 46.

1. Method to identify and quantify environmental microorganisms useful in biomining processes, wherein said method comprises the steps of:
(a) extracting DNA from a sample;
(b) quantifying the extracted DNA;
(c) optionally perform at least one primary PCR using universal primers for the kingdoms:
i. Bacteria and/or
ii. Archaea,
in order to amplify a genome region;
(d) performing a quantitative PCR (qPCR) technique, using either said DNA sample or said amplified product obtained in the corresponding primary PCR as a template, and specific primers for each taxon to be determined, where taxons are selected from:
i. Bacteria: Total bacteria, Acidiphilium sp., Leptospirillum sp., Sulfobacillus sp., Acidithiobacillus ferrooxidans and Acidithiobacillus thiooxidans; and
ii. Archaea: Total archaea, Acidianus sp., Ferroplasma sp, Metallosphaera sp, Sulfolobus sp. and Thermoplasma sp.;
(e) calculating the number of microorganisms in the sample that belong to each of the analyzed taxons.

2. Method according to claim 1, wherein DNA is quantified by spectrophotometry on step (b).

3. Method according to claim 1, wherein the primary PCR on step (c) is performed using primers for 16S rDNA gene sequences.

4. Method according to claim 1, wherein the secondary PCR on step (d) comprises a qPCR reaction for each taxon to be detected using primers that are specific for said taxon.

5. Method according to claim 3, wherein the primary PCR for the Bacteria kingdom on step (c) comprises the use of the following primers:

| | |
|---|---|
| Sense primer Eub27-F: | |
| AGA GTT TGA TCC TGG CTC AG | (SEQ ID NO:1) |
| Antisense primer Univ1492-R: | |
| GGT TAC CTT GTT ACG ACT T. | (SEQ ID NO:2) |

6. Method according to claim 3, wherein the primary PCR for the Archaea kingdom on step (c) comprises the use of the following primers:

| | |
|---|---|
| Sense primer Arch21-F: | |
| TTC CGG TTG ATC C(CT)G CCG GA | (SEQ ID NO:3) |
| Antisense primer Univ1492-R: | |
| GGT TAC CTT GTT ACG ACT T. | (SEQ ID NO:2) |

7. Method according to claim 4, wherein the primer pair to be used when the taxon to be determined is Acidiphilium sp., is the result of a combination of one of the sense primer options and one of the antisense primer options detailed as follows:

| | |
|---|---|
| sense primers: | |
| CAA CCA CGG TCG GGT CAG A | (SEQ ID NO:4) |
| GAC CTT AAG TTG ATG CGC T | (SEQ ID NO:5) |
| AGT CAA CCA CGG TCG GGT C | (SEQ ID NO:6) |
| GGT TTG ACC TTA AGT TGA TG | (SEQ ID NO:7) |
| CTT AAG TTG ATG CGC TAA C | (SEQ ID NO:8) |
| GGC AGT CAA CCA CGG TCG G | (SEQ ID NO:9) |
| CGA TGC TGA GCT GAT CCT G | (SEQ ID NO:10) |
| AAG TTG ATG CGC TAA CCG C | (SEQ ID NO:11) |
| AAA GTC GCC TAA GGA GGA G | (SEQ ID NO:12) |
| GTC GCC TAA GGA GGA GCC T | (SEQ ID NO:13) |
| AAG GAG GAG CCT GCG TCT G | (SEQ ID NO:14) |
| AGG AGC CTG CGT CTG ATT A | (SEQ ID NO:15) |
| AGG AGG CAG TCA ACC ACG GT | (SEQ ID NO:16) |
| GCG AAA GTC GCC TAA GGA G | (SEQ ID NO:17) |
| GCC TAA GGA GGA GCC TGC GT | (SEQ ID NO:18) |
| GCA AGG AGG CAG TCA ACC A | (SEQ ID NO:19) |
| GCA AGT CGC TCG GGC AGT A | (SEQ ID NO:20) |
| ACC CGT AGG AAT CTA TCC T | (SEQ ID NO:21) |
| GCA CAG TCA GGC GTG AAA TA | (SEQ ID NO:22) |
| ACA CAT GCA AGT CGC TCG GG | (SEQ ID NO:23) |
| antisense primers: | |
| TCT CTG ACC CGA CCG TGG TT | (SEQ ID NO:24) |
| TCA ACT TAA GGT CAA ACC AA | (SEQ ID NO:25) |
| GGA GCT TAT TCT GCG GGT A | (SEQ ID NO:26) |
| GCA TCA ACT TAA GGT CAA AC | (SEQ ID NO:27) |
| AGC GCA TCA ACT TAA GGT CA | (SEQ ID NO:28) |
| GTT AGC GCA TCA ACT TAA GG | (SEQ ID NO:29) |
| CCG ACC GTG GTT GAC TGC C | (SEQ ID NO:30) |
| GGA TCA GCT CAG CAT CGC TG | (SEQ ID NO:31) |
| TCA GGA TCA GCT CAG CAT CG | (SEQ ID NO:32) |
| CGG TTA GCG CAT CAA CTT A | (SEQ ID NO:33) |
| GGC TCC TCC TTA GGC GAC TT | (SEQ ID NO:34) |
| GTT GAC TGC CTC CTT GCG GT | (SEQ ID NO:35) |
| TCC TCC TTA GGC GAC TTT CG | (SEQ ID NO:36) |
| GTG GTT GAC TGC CTC CTT GC | (SEQ ID NO:37) |
| ACC GTG GTT GAC TGC CTC CT | (SEQ ID NO:38) |
| GCA GGC TCC TCC TTA GGC GA | (SEQ ID NO:39) |
| GAC GCA GGC TCC TCC TTA GG | (SEQ ID NO:40) |
| TCA GAC GCA GGC TCC TCC TT | (SEQ ID NO:41) |
| TGC TAC TGC CCG AGC GAC TT | (SEQ ID NO:42) |
| TGA CCC GAC CGT GGT TGA C | (SEQ ID NO:43) |

8. Method according to claim 4, wherein the primer pair to be used when the taxon to be determined is Leptospirillum sp., is the result of a combination of one of the sense primer options and one of the antisense primer options detailed as follows:

| | |
|---|---|
| sense primers: | |
| TGA GGG GAC TGC CAG CGA C | (SEQ ID NO:44) |
| TAA ATA TCC CCG ATG ACG G | (SEQ ID NO:45) |
| TTG TCC GGA ACC GTG AAG GG | (SEQ ID NO:46) |
| GGA ACC GTG AAG GGT TTC G | (SEQ ID NO:47) |
| CCG AAT ATT GTC CGG AAC C | (SEQ ID NO:48) |
| CGA CAG AGT TTG ATC GTG G | (SEQ ID NO:49) |
| AAT ATT GTC CGG AAC CGT G | (SEQ ID NO:50) |
| TCC GGA ACC GTG AAG GGT T | (SEQ ID NO:51) |
| AAA TCG GGC CAT CAC ACA G | (SEQ ID NO:52) |
| CAA AGA GAC TGG CAG ACT AGA | (SEQ ID NO:53) |
| TCG GGC CAT CAC ACA GGT G | (SEQ ID NO:54) |
| AGA GAC TGG CAG ACT AGA G | (SEQ ID NO:55) |
| GGG GGG GCA ATA CCG AAT AGA | (SEQ ID NO:56) |
| ATA TCA AAT AAA TAT CCC CG | (SEQ ID NO:57) |
| AAG GGA TAT CGA ATA AAT AT | (SEQ ID NO:58) |
| CTA GAG GCT GGG AGA GGG AAG | (SEQ ID NO:59) |
| GAC GCA GCA ACG CCA GCA GTG | (SEQ ID NO:60) |
| AAA TAA ATA TCC CCG ATG A | (SEQ ID NO:61) |
| CAG TGT GGG AAG AAG GCT TTC | (SEQ ID NO:62) |
| AAC AAG GTA CCC GTC TAG A | (SEQ ID NO:63) |
| antisense primers: | |
| CTA GAC GGG TAC CTT GTT AC | (SEQ ID NO:64) |
| CCG TCA TCG GGG ATA TTT A | (SEQ ID NO:65) |
| TTC ACG GTT CCG GAC AAT AT | (SEQ ID NO:66) |
| CGG TTC CGG ACA ATA TTC G | (SEQ ID NO:67) |
| CCC TTC ACG GTT CCG GAC AA | (SEQ ID NO:68) |
| CCA CGA TCA AAC TCT GTC GA | (SEQ ID NO:69) |
| AAA CCC TTC ACG GTT CCG GA | (SEQ ID NO:70) |
| TTC CGG ACA ATA TTC GGT AT | (SEQ ID NO:71) |
| CCG AAA CCC TTC ACG GTT CC | (SEQ ID NO:72) |
| TAG TCT GCC AGT CTC TTT GGC | (SEQ ID NO:73) |
| GCA CCT GTG TGA TGG CCC GAT | (SEQ ID NO:74) |
| CTC TAG TCT GCC AGT CTC TTT | (SEQ ID NO:75) |
| GCA GCA CCT GTG TGA TGG CCC | (SEQ ID NO:76) |
| CCT GTG TGA TGG CCC GAT TT | (SEQ ID NO:77) |
| TCT ATT CGG TAT TGC CCC CCC | (SEQ ID NO:78) |
| CCC CTT TCG GTT CCC TAC TCG | (SEQ ID NO:79) |
| TCC CTC TCC CAG CCT CTA GTC | (SEQ ID NO:80) |
| TCG GGG ATA TTT ATT TGA T | (SEQ ID NO:81) |
| CAT ACC TTG GGC GGC TCC CT | (SEQ ID NO:82) |
| CAG CCT CTA GTC TGC CAG T | (SEQ ID NO:83) |

9. Method according to claim 4, wherein the primer pair to be used when the taxon to be determined is Sulfobacillus sp., is the result of a combination of one of the sense primer options and one of the antisense primer options detailed as follows:

| | |
|---|---|
| sense primers: | |
| CGA AGG CGG TGC ACT GGC C | (SEQ ID NO:84) |
| GTG GCG AAG GCG GTG CAC T | (SEQ ID NO:85) |
| AGG TGT CGC GGG GGT CCA CC | (SEQ ID NO:86) |
| TGT CTG TCG GGA CGA GGA C | (SEQ ID NO:87) |
| GAG GGC AGG AGA GGT GCA T | (SEQ ID NO:88) |
| GTC CAC CTC GCG GTG CCG G | (SEQ ID NO:89) |
| CAC CTC GCG GTG CCG GAG C | (SEQ ID NO:90) |
| GGG GGT CCA CCT CGC GGT GC | (SEQ ID NO:91) |
| CTC GCG GTG CCG GAG CTA A | (SEQ ID NO:92) |
| TGT CGC GGG GGT CCA CCT C | (SEQ ID NO:93) |
| GGA TAC GAG GTG TCG CGG G | (SEQ ID NO:94) |
| CGG AGC TAA CGC ACT CAG T | (SEQ ID NO:95) |
| GTA AAC GAT GGA TAC GAG GT | (SEQ ID NO:96) |
| TGA GTG GGG GAT ATC GGG C | (SEQ ID NO:97) |
| TAC GAG GTG TCG CGG GGG T | (SEQ ID NO:98) |
| AGC TAA CGC ACT CAG TAT C | (SEQ ID NO:99) |
| ACG ATG GAT ACG AGG TGT CG | (SEQ ID NO:100) |
| GTG CCG GAG CTA ACG CAC TC | (SEQ ID NO:101) |
| AGG TGC ATG GAA TTC CTG GT | (SEQ ID NO:102) |
| TGC ATG GAA TTC CTG GTG GA | (SEQ ID NO:103) |
| antisense primers: | |
| CAG TGC ACC GCC TTC GCC A | (SEQ ID NO:104) |
| GGC CAG TGC ACC GCC TTC G | (SEQ ID NO:105) |
| GGT GGA CCC CCG CGA CAC C | (SEQ ID NO:106) |
| GGT CCT CGT CCC GAC AGA C | (SEQ ID NO:107) |
| CAT GCA CCT CTC CTG CCC TC | (SEQ ID NO:108) |
| TTA GCT CCG GCA CCG CGA GG | (SEQ ID NO:109) |
| GCG AGG TGG ACC CCC GCG A | (SEQ ID NO:110) |
| TGC ACC GCC TTC GCC ACC G | (SEQ ID NO:111) |
| CGT ATC CAT CGT TTA CGG CG | (SEQ ID NO:112) |
| GAC CCC CGC GAC ACC TCG TA | (SEQ ID NO:113) |
| GAG TGC GTT AGC TCC GGC AC | (SEQ ID NO:114) |
| TCC ACC AGG AAT TCC ATG C | (SEQ ID NO:115) |
| GCC AGG CCA GTG CAC CGC C | (SEQ ID NO:116) |
| CCA GGA ATT CCA TGC ACC TC | (SEQ ID NO:117) |
| CCT CGT ATC CAT CGT TTA CG | (SEQ ID NO:118) |
| ACT GAG TGC GTT AGC TCC GG | (SEQ ID NO:119) |
| GAT ACT GAG TGC GTT AGC TC | (SEQ ID NO:120) |
| GCG ACA CCT CGT ATC CAT CG | (SEQ ID NO:121) |
| CGG GAT ACT GAG TGC GTT AG | (SEQ ID NO:122) |
| GCC CGA TAT CCC CCA CTC A | (SEQ ID NO:123) |

10. Method according to claim 4, wherein the primer pair to be used when the taxon to be determined is Acidithiobacillus ferrooxidans, is the result of a combination of one of the sense primer options and one of the antisense primer options detailed as follows:

| | |
|---|---|
| sense primers: | |
| CGG GTT CTA ATA CAA TCT G | (SEQ ID NO:124) |
| AGG ACG AAA AGG CGG GTT CT | (SEQ ID NO:125) |
| GTG GAG GAC GAA AAG GCG G | (SEQ ID NO:126) |
| ACG AAA AGG CGG GTT CTA AT | (SEQ ID NO:127) |
| AAA AGG CGG GTT CTA ATA CA | (SEQ ID NO:128) |
| AGG CGG GTT CTA ATA CAA T | (SEQ ID NO:129) |
| TTC TAA TAC AAT CTG CTG TT | (SEQ ID NO:130) |
| TAA TAC AAT CTG CTG TTG AC | (SEQ ID NO:131) |
| TAC AAT CTG CTG TTG ACG TG | (SEQ ID NO:132) |
| AAT CTG CTG TTG ACG TGA AT | (SEQ ID NO:133) |
| CGC TAA GGG AGG AGC CTA CG | (SEQ ID NO:134) |
| GCG GAC TAG AGT ATG GGA G | (SEQ ID NO:135) |
| CTA GAG TAT GGG AGA GGG TG | (SEQ ID NO:136) |
| CCT CGC GCT AAG GGA GGA G | (SEQ ID NO:137) |
| GGC GGA CTA GAG TAT GGG AG | (SEQ ID NO:138) |
| GGG AGG AGC CTA CGT CTG AT | (SEQ ID NO:139) |
| CGC GCT AAG GGA GGA GCC T | (SEQ ID NO:140) |
| CGG ACC TCG CGC TAA GGG AG | (SEQ ID NO:141) |
| GGC GGA CTA GAG TAT GGG A | (SEQ ID NO:142) |
| TAA GGG AGG AGC CTA CGT CT | (SEQ ID NO:143) |
| antisense primers: | |
| AGA ACC CGC CTT TTC GTC CT | (SEQ ID NO:144) |
| CCG CCT TTT CGT CCT CCA C | (SEQ ID NO:145) |
| CAG ATT GTA TTA GAA CCC G | (SEQ ID NO:146) |
| ATT AGA ACC CGC CTT TTC GT | (SEQ ID NO:147) |
| TGT ATT AGA ACC CGC CTT TT | (SEQ ID NO:148) |
| CTC TGC AGA ATT CCG GAC AT | (SEQ ID NO:149) |
| AAC AGC AGA TTG TAT TAG AA | (SEQ ID NO:150) |
| GTC AAC AGC AGA TTG TAT TA | (SEQ ID NO:151) |
| CAC GTC AAC AGC AGA TTG TA | (SEQ ID NO:152) |
| ATT CAC GTC AAC AGC AGA TT | (SEQ ID NO:153) |
| GTA GGC TCC TCC CTT AGC GC | (SEQ ID NO:154) |
| GCTC CTC CCT TAG CGC GAG | (SEQ ID NO:155) |
| CCA TAC TCT AGT CCG CCG GT | (SEQ ID NO:156) |
| TCT AGT CCG CCG GTT TCC A | (SEQ ID NO:157) |
| GAC GTA GGC TCC TCC CTT AG | (SEQ ID NO:158) |
| TAC TCT AGT CCG CCG GTT T | (SEQ ID NO:159) |
| TCA GAG GTA GGC TCC TCC CT | (SEQ ID NO:160) |
| CCT CCC TTA GCG CGA GGT CC | (SEQ ID NO:161) |
| TAG TGC GCC GGT TTC CAC C | (SEQ ID NO:162) |
| ATT GTA TTA GAA CCC GCC T | (SEQ ID NO:163) |

11. Method according to claim 4, wherein the primer pair to be used when the taxon to be determined is Acidithiobacillus thiooxidans, is the result of a combination of one of the sense primer options and one of the antisense primer options detailed as follows:

| | |
|---|---|
| sense primers: | |
| GGG AGA CGA AAA GGT AAT CG | (SEQ ID NO:164) |
| AAA GTT CTT TCG GTG ACG GG | (SEQ ID NO:165) |
| CGG GGA AGG TTG ATA TGT TA | (SEQ ID NO:166) |
| GAG GGA GAA ACC GGG GGA T | (SEQ ID NO:167) |
| AAT CGC TAA TAT CGG TTA C | (SEQ ID NO:168) |
| CCG GGG GAT CTT CGG ACC TC | (SEQ ID NO:169) |
| TAA TAT CGCC TGC TGT TGA C | (SEQ ID NO:170) |
| TCG GTG ACG GGG AAG GTT G | (SEQ ID NO:171) |
| GGA GAA ACC GGG GGA TCT T | (SEQ ID NO:172) |
| ACG TCC TGA GGG AGA AAC CG | (SEQ ID NO:173) |
| AGA CGA AAA GGT AAT CGC TA | (SEQ ID NO:174) |
| GTG ACG GGG AAG GTT GAT A | (SEQ ID NO:175) |
| GAA ACC GGG GGA TCT TCG G | (SEQ ID NO:176) |
| TCC TGA GGG AGA AAC CGG GG | (SEQ ID NO:177) |
| CGA AAA GGT AAT CGC TAA TA | (SEQ ID NO:178) |
| AAA GGT AAT CGC TAA TAT CG | (SEQ ID NO:179) |
| TCG TGG GAG ACG AAA AGG TA | (SEQ ID NO:180) |
| CGG ACC TCG TGC TAT TGG AG | (SEQ ID NO:181) |
| GTT CTT TCG GTG ACG GGG A | (SEQ ID NO:182) |
| CTT TCG GTG ACG GGG AAG G | (SEQ ID NO:183) |
| antisense primers: | |
| ATC CCC CGG TTT CTC CCT C | (SEQ ID NO:184) |
| ATA TTA GCG ATT ACC TTT T | (SEQ ID NO:185) |
| CAA CCT TCC CCG TCA CCG AA | (SEQ ID NO:186) |
| CCG AAG ATC CCC CGG TTT CT | (SEQ ID NO:187) |
| CTC CAA TAG CAC GAG GTC CG | (SEQ ID NO:188) |
| ACC GAT ATT AGC GAT TAC CT | (SEQ ID NO:189) |
| AAG ATC CCC CGG TTT CTC C | (SEQ ID NO:190) |
| TAT CAA CCT TCC CCG TCA CC | (SEQ ID NO:191) |
| GGT TTC TCC CTC AGG ACG TA | (SEQ ID NO:192) |
| GGT CCG AAG ATC CCC CGG TT | (SEQ ID NO:193) |
| TTT CAC GAC AGA CCT AAT G | (SEQ ID NO:194) |
| GTA ACC GAT ATT AGC GAT TA | (SEQ ID NO:195) |
| ACA TAT CAA CCT TCC CCG TC | (SEQ ID NO:196) |
| CCC GGT TTC TCC CTC AGG AC | (SEQ ID NO:197) |
| GCG ATT ACC TTT TCG TCT CC | (SEQ ID NO:198) |
| CCC CGT CAC CGA AAG AAC TT | (SEQ ID NO:199) |
| TTA ACA TAT CAA CCT TCC CC | (SEQ ID NO:200) |
| TTA GCG ATT ACC TTT TCG TC | (SEQ ID NO:201) |
| CTT CCC CGT CAC CGA AAG AA | (SEQ ID NO:202) |
| ATT ACC TTT TCG TCT CCC AC | (SEQ ID NO:203) |

12. Method according to claim 4, wherein the primer pair to be used when the taxon to be determined is Acidianus sp., is the result of a combination of one of the sense primer options and one of the antisense primer options detailed as follows:

| | |
|---|---|
| sense primers: | |
| GGG AAA CCG TGA GGG CGC T | (SEQ ID NO:204) |
| GCG AAA CGT CCC CAA TGC GG | (SEQ ID NO:205) |
| CCG CAG GGA AAC CGG TAA GCC | (SEQ ID NO:206) |
| CCC GGG AAA GGG CAG TGA TA | (SEQ ID NO:207) |
| GGG AAA GGG CAG TGA TAC T | (SEQ ID NO:208) |
| AAT CCG GGG CAG GCG AAC GG | (SEQ ID NO:209) |
| AGG GTA CTG GAA CGT CCC TT | (SEQ ID NO:210) |
| AAG CGT CCG GCC AGA ACG CGC | (SEQ ID NO:211) |
| CGC CTA AAG GGG CAT GGG CT | (SEQ ID NO:212) |
| GGC TAT TTC CCG CTC ATC CC | (SEQ ID NO:213) |
| CGT ACG CCC TCG GGT AAG AGG | (SEQ ID NO:214) |
| AAC GGC CCG CCA AAC CGA TA | (SEQ ID NO:215) |
| AGC CGG CCC TGC AAG TCA C | (SEQ ID NO:216) |
| CAC TGC TTA AAG ACC CGG G | (SEQ ID NO:217) |
| GGA GCT AAT CCG GGG CAG GCG | (SEQ ID NO:218) |
| AAA CCG TGA GGG CGC TAC CC | (SEQ ID NO:219) |
| AGG CGA AGG GTA CTG GAA CGT | (SEQ ID NO:220) |
| ACC CCC AGT GCT CCC GAA AG | (SEQ ID NO:221) |
| CCC TTC GCC TAA AGG GGC ATG | (SEQ ID NO:222) |
| GCA TGG GCT ATT TCC CGC TCA | (SEQ ID NO:223) |
| antisense primers: | |
| CCG CAT TGG GGA CGT TTC GCG | (SEQ ID NO:226) |
| GCG CCC TCA CGG TTT CCC GCA | (SEQ ID NO:227) |
| CCG CAT TGG GGA CGT TTC GCG | (SEQ ID NO:228) |
| GCG CCC TCA CGG TTT CCC GCA | (SEQ ID NO:229) |
| TTC CCG CAT TGG GGA CGT TTC | (SEQ ID NO:230) |
| TAG CGC CCT CAC GGT TTC CC | (SEQ ID NO:231) |
| GGC TTA CCG GTT TCC CTG CG | (SEQ ID NO:232) |
| CTG CCC TTT CCC GGG TTG A | (SEQ ID NO:233) |
| TCA CTG CCC TTT CCC GGG T | (SEQ ID NO:234) |
| GTA TCA CTG CCC TTT CCC G | (SEQ ID NO:235) |
| GCC CGG GTC TTT AAG CAG TG | (SEQ ID NO:236) |
| CTC CCG CCC CCT AGC CCT GCA | (SEQ ID NO:237) |
| CCC GGG ATC TGT GGA TTT CGC | (SEQ ID NO:238) |
| TAC CCG AGG GCG TAC GAC T | (SEQ ID NO:239) |
| CCT CTT ACC CGA GGG CGT ACG | (SEQ ID NO:240) |
| TTC GCC TGC CCC GGA TTA G | (SEQ ID NO:241) |
| GGC GGC AGG CTT ACC GGT TTC | (SEQ ID NO:242) |
| CGG ATT AGC TCC AGT TTC CCG | (SEQ ID NO:243) |
| GGA CGT TCC AGT ACC CTT C | (SEQ ID NO:244) |
| CCC CGG ATT AGC TCC AGT TT | (SEQ ID NO:245) |

13. Method according to claim 4, wherein the primer pair to be used when the taxon to be determined is Ferroplasma sp., is the result of a combination of one of the sense primer options and one of the antisense primer options detailed as follows:

| | |
|---|---|
| sense primers: | |
| AGA GTC AAC CTG ACG AGC TTA | (SEQ ID NO:248) |
| GTC AAC CTG ACG AGC TTA CTC | (SEQ ID NO:249) |
| TGA GAG TCA ACC TGA CGA GC | (SEQ ID NO:250) |
| GAG CTT ACT CGA TAG CAG GAG | (SEQ ID NO:251) |
| TTT AAT TCG AGA GGG TTA A | (SEQ ID NO:252) |
| CTT ACT CGA TAG CAG GAG AGG | (SEQ ID NO:253) |
| AAT CAA ATC TGA TGT CGG TGA | (SEQ ID NO:254) |
| GGT TAA ATC AAA TCT GAT G | (SEQ ID NO:255) |
| TTC GAG AGG GTT AAA TCA AAT | (SEQ ID NO:256) |
| CAA ATC TGA TGT CGG TGA GGA | (SEQ ID NO:257) |
| TAA ATC AAA TCT GAT GTC G | (SEQ ID NO:258) |
| GAG AGG GTT AAA TCA AAT CTG | (SEQ ID NO:259) |
| ATC TGA TGT CGG TGA GGA GGG | (SEQ ID NO:260) |
| AAT TCG AGA GGG TTA AAT C | (SEQ ID NO:261) |
| GAT GTC GGT GAG GAG GGT T | (SEQ ID NO:262) |
| GAG GGA TGG CAG TGT CGG A | (SEQ ID NO:263) |
| TGG CCA AGA CTT TTC TCA T | (SEQ ID NO:264) |
| GAT GAG TCT GCA ACC TAT CA | (SEQ ID NO:265) |
| TAG CAG AGA GGT GGT GCA TGG | (SEQ ID NO:266) |
| ACG GCC ACT GCT ATC AAG TTC | (SEQ ID NO:267) |
| antisense primers: | |
| AAG CTC GTC AGG TTG ACT CT | (SEQ ID NO:268) |
| GTA AGC TCG TCA GGT TGA C | (SEQ ID NO:269) |
| CGA GTA AGC TCG TCA GGT T | (SEQ ID NO:270) |
| CTG CTA TCG AGT AAG CTC G | (SEQ ID NO:271) |
| TTT AAC CCT CTC GAA TTA A | (SEQ ID NO:272) |
| CTC CTG CTA TCG AGT AAG C | (SEQ ID NO:273) |
| TCA GAT TTG ATT TAA CCC TC | (SEQ ID NO:274) |
| ACC CTC CTC ACC GAC ATC AG | (SEQ ID NO:275) |
| ACA TCA GAT TTG ATT TAA C | (SEQ ID NO:276) |
| CCG ACA TCA GAT TTG ATT T | (SEQ ID NO:277) |
| TGA TTT AAC CCT CTC GAA T | (SEQ ID NO:278) |
| TCA CCG ACA TCA GAT TTG A | (SEQ ID NO:279) |
| ATT TGA TTT AAC CCT CTC G | (SEQ ID NO:280) |
| CTA CCT GAT AGG TTG CAG ACT | (SEQ ID NO:281) |
| GCA CCA CCT CTC TGC TAT CG | (SEQ ID NO:282) |
| ATC CCT CAA CGG AAA AGC A | (SEQ ID NO:283) |
| ACA CTT AAA GTG AAC GCC CT | (SEQ ID NO:284) |
| TCG CTC CGA CAC TGC CAT C | (SEQ ID NO:285) |
| CCG ATC TCA TGT CTT GCA GT | (SEQ ID NO:286) |
| ATG AGA AAA GTC TTG GCC A | (SEQ ID NO:287) |

14. Method according to claim 4, wherein the primer pair to be used when the taxon to be determined is Metallosphaera sp., is the result of a combination of one of the sense primer options and one of the antisense primer options detailed as follows:

| | |
|---|---|
| sense primers: | |
| AGG GCG TTA CCC CTA GTG C | (SEQ ID NO:288) |
| TAC CCC TAG TGC CCT CGC A | (SEQ ID NO:289) |
| GCG CCC GTA GCC GGC CTG TAA | (SEQ ID NO:290) |
| GAG CTT CTC CTC CGC GAG GGG | (SEQ ID NO:291) |
| GCA CCA GGC GCG GAA CGT CCC | (SEQ ID NO:292) |
| GAG GTC GAG CTT CTC CTC CG | (SEQ ID NO:293) |
| CCC TAG TGC CCT CGC AAG A | (SEQ ID NO:294) |
| CCC GTA GCC GGC CTG TAA AGT | (SEQ ID NO:295) |
| CGG GGT GGG AGG TCG AGC TTC | (SEQ ID NO:296) |
| GTC GAG CTT CTC CTC CGC GA | (SEQ ID NO:297) |
| GGT GGG AGG TCG AGC TTC TCC | (SEQ ID NO:298) |
| TCG GGG TGG GAG GTC GAG C | (SEQ ID NO:299) |
| GCG TTA CCC CTA GTG CCC T | (SEQ ID NO:300) |
| TAG GGG TAG GGC TAA GCC ATG | (SEQ ID NO:301) |
| CGC ACC AGG CGC GGA ACG T | (SEQ ID NO:302) |
| GGG AGG TCG AGC TTC TCC T | (SEQ ID NO:303) |
| AGG TGG AGG AAT AAG CGG GG | (SEQ ID NO:304) |
| GAA AGG TGG AGG AAT AAG C | (SEQ ID NO:305) |
| GGG AGT CGT ACG CTC TCG GGA | (SEQ ID NO:306) |
| CTA ACC TGC CCT TGG GAT CTG | (SEQ ID NO:307) |
| antisense primers: | |
| GGC ACT AGG GGT AAC GCC C | (SEQ ID NO:308) |
| AGA AGC TCG ACC TCC CAC CC | (SEQ ID NO:309) |
| TAC AGG CCG GCT ACG GGC GC | (SEQ ID NO:310) |
| AGC TCG ACC TCC CAC CCC G | (SEQ ID NO:311) |
| CCC CTC GCG GAG GAG AAG C | (SEQ ID NO:312) |
| TGC GAG GGC ACT AGG GGT A | (SEQ ID NO:313) |
| TGA CTT TAC AGG CCG GCT ACG | (SEQ ID NO:314) |
| CAT GGC TTA GCC CTA CCC CTA | (SEQ ID NO:315) |
| AGG AGA AGC TCG ACC TCC CA | (SEQ ID NO:316) |
| GAC GTT CCG CGC CTG GTG C | (SEQ ID NO:317) |
| CTT TAC AGG CCG GCT ACG GG | (SEQ ID NO:318) |
| TCT TGC GAG GGC ACT AGG G | (SEQ ID NO:319) |
| CGG AGG AGA AGC TCG ACC TC | (SEQ ID NO:320) |
| TCG CGG AGG AGA AGC TCG AC | (SEQ ID NO:321) |
| GAG GGC ACT AGG GGT AAC G | (SEQ ID NO:322) |
| ACC CCG AGG GGC AAG AGG CC | (SEQ ID NO:323) |
| GGG GTT ATC CAG ATC CCA AGG | (SEQ ID NO:324) |
| GCC ACG CCC TCT TCC CGA GA | (SEQ ID NO:325) |
| GTT ATC CAG ATC CCA AGG GC | (SEQ ID NO:326) |
| CTT ATT CCT CCA CCT TTC TGG | (SEQ ID NO:327) |

15. Method according to claim 4, wherein the primer pair to be used when the taxon to be determined is Sulfolobus sp., is the result of a combination of one of the sense primer options and one of the antisense primer options detailed as follows:

| | |
|---|---|
| sense primers: | |
| TAA ACC CTG CCG CAG TTG G | (SEQ ID NO:328) |
| CCT TAA ACC CTG CCG CAG T | (SEQ ID NO:329) |
| GTC CTG GAA CGG TTC CTC G | (SEQ ID NO:330) |
| CTC TAC AAA GGC GGG GGA ATA | (SEQ ID NO:331) |
| CTG GAA CGG TTC CTC GCT GA | (SEQ ID NO:332) |
| GGC GAG GAG TCC TGG AAC GGT | (SEQ ID NO:333) |
| TTT CCC CGC TCT ACA AAG G | (SEQ ID NO:334) |
| TAC AAA GGC GGG GGA ATA AGC | (SEQ ID NO:335) |
| CGC TCT ACA AAG GCG GGG G | (SEQ ID NO:336) |
| ATA GGC GAG GAG TCC TGG AA | (SEQ ID NO:337) |
| CCA TAG GCG AGG AGT CCT G | (SEQ ID NO:338) |
| GCT TTT CCC CGC TCT ACA A | (SEQ ID NO:339) |
| GCT AAC CTA CCC TGA GGA GG | (SEQ ID NO:340) |
| TCT CCC ATA GGC GAG GAG TC | (SEQ ID NO:341) |
| TGG CTA ACC TAC CCT GAG G | (SEQ ID NO:342) |
| ATA ATC TCC CAT AGG CGA G | (SEQ ID NO:343) |
| TGA GGA GGG AGA TAA CCC CG | (SEQ ID NO:344) |
| ACA CGT GGC TAA CCT ACC CTG | (SEQ ID NO:345) |
| CCT GAG GAG GGA GAT AAC C | (SEQ ID NO:346) |
| AAA CTG GGG ATA ATC TCC C | (SEQ ID NO:347) |
| antisense primers: | |
| CCA ACT GCG GCA GGG TTT A | (SEQ ID NO:348) |
| ACT GCG GCA GGG TTT AAG G | (SEQ ID NO:349) |
| CGA GGA ACC GTT CCA GGA CTC | (SEQ ID NO:350) |
| AAC CGT TCC AGG ACT CCT CG | (SEQ ID NO:351) |
| TCC AGG ACT CCT CGC CTA TGG | (SEQ ID NO:352) |
| CCT TTG TAG AGC GGG GAA A | (SEQ ID NO:353) |
| AGC GAG GAA CCG TTC CAG GA | (SEQ ID NO:354) |
| CGT TCC AGG ACT CCT CGC CTA | (SEQ ID NO:355) |
| CCC CCG CCT TTG TAG AGC G | (SEQ ID NO:356) |
| TTC AGC GAG GAA CCG TTC CA | (SEQ ID NO:357) |
| ATT CCC CCG CCT TTG TAG A | (SEQ ID NO:358) |
| TTG TAG AGC GGG GAA AAG C | (SEQ ID NO:359) |
| ATC TCC CTC CTC AGG GTA GGT | (SEQ ID NO:360) |
| GGG TTA TCT CCC TCC TCA G | (SEQ ID NO:361) |
| TCG CCT ATG GGA GAT TAT C | (SEQ ID NO:362) |
| TCA GGG TAG GTT AGC CAC GT | (SEQ ID NO:363) |
| CCT CAG GGT AGG TTA GCC A | (SEQ ID NO:364) |
| CCG GGG TTA TCT CCC TCC T | (SEQ ID NO:365) |
| TCC TCG CCT ATG GGA GAT T | (SEQ ID NO:366) |
| CCT CCT CAG GGT AGG TTA G | (SEQ ID NO:367) |

16. Method according to claim 4, wherein the primer pair to be used when the taxon to be determined is Thermoplasma sp., is the result of a combination of one of the sense primer options and one of the antisense primer options detailed as follows:

| | |
|---|---|
| sense primers: | |
| TCC TGA AAG GAC GAC CGG TG | (SEO ID NO:368) |
| GGA CTG AGG GCT GTA ACT C | (SEQ ID NO:369) |
| GAG GTT GAA TGT ACT TTC AGG | (SEQ ID NO:370) |
| GGT GGC GAA AGC GTT CAA CT | (SEQ ID NO:371) |
| GCC CTC ACG AAT GTG GAT T | (SEQ ID NO:372) |
| ACC TCG AAA CCC GTT CGT AG | (SEQ ID NO:373) |
| TCC GTA GTA ATC GTA GGT C | (SEQ ID NO:374) |
| ATC CTG TAA TCC TGA AAG GAC | (SEQ ID NO:375) |
| GTA GTC AGG ACT GAG GGC TG | (SEQ ID NO:376) |
| AGG ACG ACC GGT GGC GAA AGC | (SEQ ID NO:377) |
| TAA CTC GCC CTC ACG AAT GT | (SEQ ID NO:378) |
| GAA GGT GTT AAG TGG GTC A | (SEQ ID NO:379) |
| AAA CCC GTT CGT AGT CAG GAC | (SEQ ID NO:380) |
| TAC GGT GAA TAT GCC CCT GC | (SEQ ID NO:381) |
| CAC TTG GTG TTG CTT CTC CGT | (SEQ ID NO:382) |
| GAT CAC TTT TAT TGA GTC T | (SEQ ID NO:383) |
| AGC ATC AGG AAT AAG GGC TG | (SEQ ID NO:384) |
| AAG ACC CCC ATC TCT AAT T | (SEQ ID NO:385) |
| CCG GTC TTA TAA ATC TTC A | (SEQ ID NO:386) |
| ATA ACG AGC AAG ACC CCC AT | (SEQ ID NO:387) |
| antisense primers: | |
| CAG GGG CAT ATT CAC CGT AG | (SEQ ID NO:388) |
| TCA GGA TTA CAG GAT TTT A | (SEQ ID NO:389) |
| ACC CTG AAA GTA CAT TCA ACC | (SEQ ID NO:390) |
| GCC ACC GGT CGT CCT TTC A | (SEQ ID NO:391) |
| CTA GTT GAA CGC TTT CGC C | (SEQ ID NO:392) |
| TCG TCC TTT CAG GAT TAC AGG | (SEQ ID NO:393) |
| ACG CTT TCG CCA CCG GTC GTC | (SEQ ID NO:394) |
| GGG TTT CGA GGT TAG CTT C | (SEQ ID NO:395) |
| CCC TCA GTC CTG ACT ACG A | (SEQ ID NO:396) |
| CTG AAG ATT TAT AAG ACC GG | (SEQ ID NO:397) |
| TTA CAG CCC TCA GTC CTG ACT | (SEQ ID NO:398) |
| AAT CCA CAT TCG TGA GGG CGA | (SEQ ID NO:399) |
| ATG GGG GTC TTG CTC GTT AT | (SEQ ID NO:400) |
| GCT GTT GAC CTA CGA TTA C | (SEQ ID NO:401) |
| CCT ACG ATT ACT ACG GAA TCC | (SEQ ID NO:402) |
| ACC CAC TTA ACA CCT TCG C | (SEQ ID NO:403) |
| CCC AAG TCT TAC AGT CTC TT | (SEQ ID NO:404) |
| CTA CCC TGA AAG TAC ATT CA | (SEQ ID NO:405) |
| CAG CCC TTA TTC CTG ATG C | (SEQ ID NO:406) |
| GGT CGT CCT TTC AGG ATT AC | (SEQ ID NO:407) |

17. Method according to claim 4, wherein said method comprises a qPCR reaction to determine total Bacteria and/or a qPCR reaction to determine total Archaea, wherein used primers are known and are selected from a combination of one of the sense primer options and one of the antisense primer options for each kingdom, as detailed as follows:
Seq ID Nos.
Bacteria primers

| | | |
|---|---|---|
| Eub27 F | AGA GTT TGA TCC TGG | (SEQ ID NO:1) |
| | CTC AG | |
| Univ533-F | GTG CCA GCM GCC GCG | (SEQ ID NO:408) |
| | GTA | |
| Bact358-F | CCT ACG GGA GGC AGC AG | (SEQ ID NO:409) |
| Univ907-R | CCG TCA ATT CCT TTG | (SEQ ID NO:410) |
| | AGT T | |
| Bact338-R | GCT GCC TCC CGT AGG | (SEQ ID NO:411) |
| | AGT | |
| Bact1387-R | GGG CGG WGT GTA CAA | (SEQ ID NO:412) |
| | GGC | |

Archaea primers

| | | |
|---|---|---|
| Arch344-F | ACG GGG CGC AGC AGG | (SEQ ID NO:413) |
| | CGC GA | |
| Univ515-F | GTG CCA GCA GCC GCG | (SEQ ID NO:414) |
| | GTA A | |
| Arch958-R | YCC GGC GTT GAM TCC | (SEQ ID NO:415) |
| | AAT T | |
| Arch915-R | GTG CTC CCC CGC CAA | (SEQ ID NO:416) |
| | TTC CT | |
| Univ534-R | ATT ACC GCG GCT GCT | (SEQ ID NO:417) |
| | GG | |

18. Method according to claim 4, wherein qPCR results are stored on step (d) and the initial DNA concentration in each reaction sample (Q) is determined.

19. Method according to claim 1, wherein the number of microorganisms in said biomining sample is calculated using the following mathematical formula:
$$Mo / Um = \frac{Q \times T}{5 \cdot {10}^{- 6} [ ng \text{/} mo ] \times U \times Cm}$$
where:
Mo/Um is the number of microorganisms, either bacteria or archaea, per sample unit;
Q is the amount of initial DNA in nanograms that is present in each secondary PCR reaction, as determined by the program associated to the qPCR equipment;
T is the amount of total DNA extracted from the sample;
U is the amount of DNA used in the primary PCR reaction; and
Cₘ is the amount of biomining sample from which DNA was extracted, which is conveniently expressed in ml for liquid samples or in g for solid samples.


SEQUENCE LISTING (417 sequences)

SEQ ID NO: 1 - DNA, 20 bp, Artificial Sequence
  PCR primer
agagtttgatcctggctcag

SEQ ID NO: 2 - DNA, 19 bp, Artificial Sequence
  PCR primer
ggttaccttgttacgactt

SEQ ID NO: 3 - DNA, 20 bp, Artificial Sequence
  PCR primer
  misc_feature (14)..(14): n is c or t
ttccggttgatccngccgga

SEQ ID NO: 4 - DNA, 19 bp, Artificial Sequence
  Sense primer
caaccacggtcgggtcaga

SEQ ID NO: 5 - DNA, 19 bp, Artificial Sequence
  Sense primer
gaccttaagttgatgcgct

SEQ ID NO: 6 - DNA, 19 bp, Artificial Sequence
  Sense primer
agtcaaccacggtcgggtc

SEQ ID NO: 7 - DNA, 20 bp, Artificial Sequence
  Sense primer
ggtttgaccttaagttgatg

SEQ ID NO: 8 - DNA, 19 bp, Artificial Sequence
  Sense primer
cttaagttgatgcgctaac

SEQ ID NO: 9 - DNA, 19 bp, Artificial Sequence
  Sense primer
ggcagtcaaccacggtcgg

SEQ ID NO: 10 - DNA, 19 bp, Artificial Sequence
  Sense primer
cgatgctgagctgatcctg

SEQ ID NO: 11 - DNA, 19 bp, Artificial Sequence
  Sense primer
aagttgatgcgctaaccgc

SEQ ID NO: 12 - DNA, 19 bp, Artificial Sequence
  Sense primer
aaagtcgcctaaggaggag

SEQ ID NO: 13 - DNA, 19 bp, Artificial Sequence
  Sense primer
gtcgcctaaggaggagcct

SEQ ID NO: 14 - DNA, 19 bp, Artificial Sequence
  Sense primer
aaggaggagcctgcgtctg

SEQ ID NO: 15 - DNA, 19 bp, Artificial Sequence
  Sense primer
aggagcctgcgtctgatta

SEQ ID NO: 16 - DNA, 20 bp, Artificial Sequence
  Sense primer
aggaggcagtcaaccacggt

SEQ ID NO: 17 - DNA, 19 bp, Artificial Sequence
  Sense primer
gcgaaagtcgcctaaggag

SEQ ID NO: 18 - DNA, 20 bp, Artificial Sequence
  Sense primer
gcctaaggaggagcctgcgt

SEQ ID NO: 19 - DNA, 19 bp, Artificial Sequence
  Sense primer
gcaaggaggcagtcaacca

SEQ ID NO: 20 - DNA, 19 bp, Artificial Sequence
  Sense primer
gcaagtcgctcgggcagta

SEQ ID NO: 21 - DNA, 19 bp, Artificial Sequence
  Sense primer
acccgtaggaatctatcct

SEQ ID NO: 22 - DNA, 20 bp, Artificial Sequence
  Sense primer
gcacagtcaggcgtgaaata

SEQ ID NO: 23 - DNA, 20 bp, Artificial Sequence
  Sense primer
acacatgcaagtcgctcggg

SEQ ID NO: 24 - DNA, 20 bp, Artificial Sequence
  Antisense primer
tctctgacccgaccgtggtt

SEQ ID NO: 25 - DNA, 20 bp, Artificial Sequence
  Antisense primer
tcaacttaaggtcaaaccaa

SEQ ID NO: 26 - DNA, 19 bp, Artificial Sequence
  Antisense primer
ggagcttattctgcgggta

SEQ ID NO: 27 - DNA, 20 bp, Artificial Sequence
  Antisense primer
gcatcaacttaaggtcaaac

SEQ ID NO: 28 - DNA, 20 bp, Artificial Sequence
  Antisense primer
agcgcatcaacttaaggtca

SEQ ID NO: 29 - DNA, 20 bp, Artificial Sequence
  Antisense primer
gttagcgcatcaacttaagg

SEQ ID NO: 30 - DNA, 19 bp, Artificial Sequence
  Antisense primer
ccgaccgtggttgactgcc

SEQ ID NO: 31 - DNA, 20 bp, Artificial Sequence
  Antisense primer
ggatcagctcagcatcgctg

SEQ ID NO: 32 - DNA, 20 bp, Artificial Sequence
  Antisense primer
tcaggatcagctcagcatcg

SEQ ID NO: 33 - DNA, 19 bp, Artificial Sequence
  Antisense primer
cggttagcgcatcaactta

SEQ ID NO: 34 - DNA, 20 bp, Artificial Sequence
  Antisense primer
ggctcctccttaggcgactt

SEQ ID NO: 35 - DNA, 20 bp, Artificial Sequence
  Antisense primer
gttgactgcctccttgcggt

SEQ ID NO: 36 - DNA, 20 bp, Artificial Sequence
  Antisense primer
tcctccttaggcgactttcg

SEQ ID NO: 37 - DNA, 20 bp, Artificial Sequence
  Antisense primer
gtggttgactgcctccttgc

SEQ ID NO: 38 - DNA, 20 bp, Artificial Sequence
  Antisense primer
accgtggttgactgcctcct

SEQ ID NO: 39 - DNA, 20 bp, Artificial Sequence
  Antisense primer
gcaggctcctccttaggcga

SEQ ID NO: 40 - DNA, 20 bp, Artificial Sequence
  Antisense primer
gacgcaggctcctccttagg

SEQ ID NO: 41 - DNA, 20 bp, Artificial Sequence
  Antisense primer
tcagacgcaggctcctcctt

SEQ ID NO: 42 - DNA, 20 bp, Artificial Sequence
  Antisense primer
tgctactgcccgagcgactt

SEQ ID NO: 43 - DNA, 19 bp, Artificial Sequence
  Antisense primer
tgacccgaccgtggttgac

SEQ ID NO: 44 - DNA, 19 bp, Artificial Sequence
  Sense primer
tgaggggactgccagcgac

SEQ ID NO: 45 - DNA, 19 bp, Artificial Sequence
  Sense primer
taaatatccccgatgacgg

SEQ ID NO: 46 - DNA, 20 bp, Artificial Sequence
  Sense primer
ttgtccggaaccgtgaaggg

SEQ ID NO: 47 - DNA, 19 bp, Artificial Sequence
  Sense primer
ggaaccgtgaagggtttcg

SEQ ID NO: 48 - DNA, 19 bp, Artificial Sequence
  Sense primer
ccgaatattgtccggaacc

SEQ ID NO: 49 - DNA, 19 bp, Artificial Sequence
  Sense primer
cgacagagtttgatcgtgg

SEQ ID NO: 50 - DNA, 19 bp, Artificial Sequence
  Sense primer
aatattgtccggaaccgtg

SEQ ID NO: 51 - DNA, 19 bp, Artificial Sequence
  Sense primer
tccggaaccgtgaagggtt

SEQ ID NO: 52 - DNA, 19 bp, Artificial Sequence
  Sense primer
aaatcgggccatcacacag

SEQ ID NO: 53 - DNA, 21 bp, Artificial Sequence
  Sense primer
caaagagactggcagactaga

SEQ ID NO: 54 - DNA, 19 bp, Artificial Sequence
  Sense primer
tcgggccatcacacaggtg

SEQ ID NO: 55 - DNA, 19 bp, Artificial Sequence
  Sense primer
agagactggcagactagag

SEQ ID NO: 56 - DNA, 21 bp, Artificial Sequence
  Sense primer
gggggggcaataccgaataga

SEQ ID NO: 57 - DNA, 20 bp, Artificial Sequence
  Sense primer
atatcaaataaatatccccg

SEQ ID NO: 58 - DNA, 20 bp, Artificial Sequence
  Sense primer
aagggatatcgaataaatat

SEQ ID NO: 59 - DNA, 21 bp, Artificial Sequence
  Sense primer
ctagaggctgggagagggaag

SEQ ID NO: 60 - DNA, 21 bp, Artificial Sequence
  Sense primer
gacgcagcaacgccagcagtg

SEQ ID NO: 61 - DNA, 19 bp, Artificial Sequence
  Sense primer
aaataaatatccccgatga

SEQ ID NO: 62 - DNA, 21 bp, Artificial Sequence
  Sense primer
cagtgtgggaagaaggctttc

SEQ ID NO: 63 - DNA, 19 bp, Artificial Sequence
  Sense primer
aacaaggtacccgtctaga

SEQ ID NO: 64 - DNA, 20 bp, Artificial Sequence
  Antisense primer
ctagacgggtaccttgttac

SEQ ID NO: 65 - DNA, 19 bp, Artificial Sequence
  Antisense primer
ccgtcatcggggatattta

SEQ ID NO: 66 - DNA, 20 bp, Artificial Sequence
  Antisense primer
ttcacggttccggacaatat

SEQ ID NO: 67 - DNA, 19 bp, Artificial Sequence
  Antisense primer
cggttccggacaatattcg

SEQ ID NO: 68 - DNA, 20 bp, Artificial Sequence
  Antisense primer
cccttcacggttccggacaa

SEQ ID NO: 69 - DNA, 20 bp, Artificial Sequence
  Antisense primer
ccacgatcaaactctgtcga

SEQ ID NO: 70 - DNA, 20 bp, Artificial Sequence
  Antisense primer
aaacccttcacggttccgga

SEQ ID NO: 71 - DNA, 20 bp, Artificial Sequence
  Antisense primer
ttccggacaatattcggtat

SEQ ID NO: 72 - DNA, 20 bp, Artificial Sequence
  Antisense primer
ccgaaacccttcacggttcc

SEQ ID NO: 73 - DNA, 21 bp, Artificial Sequence
  Antisense primer
tagtctgccagtctctttggc

SEQ ID NO: 74 - DNA, 21 bp, Artificial Sequence
  Antisense primer
gcacctgtgtgatggcccgat

SEQ ID NO: 75 - DNA, 21 bp, Artificial Sequence
  Antisense primer
ctctagtctgccagtctcttt

SEQ ID NO: 76 - DNA, 21 bp, Artificial Sequence
  Antisense primer
gcagcacctgtgtgatggccc

SEQ ID NO: 77 - DNA, 20 bp, Artificial Sequence
  Antisense primer
cctgtgtgatggcccgattt

SEQ ID NO: 78 - DNA, 21 bp, Artificial Sequence
  Antisense primer
tctattcggtattgccccccc

SEQ ID NO: 79 - DNA, 21 bp, Artificial Sequence
  Antisense primer
cccctttcggttccctactcg

SEQ ID NO: 80 - DNA, 21 bp, Artificial Sequence
  Antisense primer
tccctctcccagcctctagtc

SEQ ID NO: 81 - DNA, 19 bp, Artificial Sequence
  Antisense primer
tcggggatatttatttgat

SEQ ID NO: 82 - DNA, 20 bp, Artificial Sequence
  Antisense primer
cataccttgggcggctccct

SEQ ID NO: 83 - DNA, 19 bp, Artificial Sequence
  Antisense primer
cagcctctagtctgccagt

SEQ ID NO: 84 - DNA, 19 bp, Artificial Sequence
  Sense primer
cgaaggcggtgcactggcc

SEQ ID NO: 85 - DNA, 19 bp, Artificial Sequence
  Sense primer
gtggcgaaggcggtgcact

SEQ ID NO: 86 - DNA, 20 bp, Artificial Sequence
  Sense primer
aggtgtcgcgggggtccacc

SEQ ID NO: 87 - DNA, 19 bp, Artificial Sequence
  Sense primer
tgtctgtcgggacgaggac

SEQ ID NO: 88 - DNA, 19 bp, Artificial Sequence
  Sense primer
gagggcaggagaggtgcat

SEQ ID NO: 89 - DNA, 19 bp, Artificial Sequence
  Sense primer
gtccacctcgcggtgccgg

SEQ ID NO: 90 - DNA, 19 bp, Artificial Sequence
  Sense primer
cacctcgcggtgccggagc

SEQ ID NO: 91 - DNA, 20 bp, Artificial Sequence
  Sense primer
gggggtccacctcgcggtgc

SEQ ID NO: 92 - DNA, 19 bp, Artificial Sequence
  Sense primer
ctcgcggtgccggagctaa

SEQ ID NO: 93 - DNA, 19 bp, Artificial Sequence
  Sense primer
tgtcgcgggggtccacctc

SEQ ID NO: 94 - DNA, 19 bp, Artificial Sequence
  Sense primer
ggatacgaggtgtcgcggg

SEQ ID NO: 95 - DNA, 19 bp, Artificial Sequence
  Sense primer
cggagctaacgcactcagt

SEQ ID NO: 96 - DNA, 20 bp, Artificial Sequence
  Sense primer
gtaaacgatggatacgaggt

SEQ ID NO: 97 - DNA, 19 bp, Artificial Sequence
  Sense primer
tgagtgggggatatcgggc

SEQ ID NO: 98 - DNA, 19 bp, Artificial Sequence
  Sense primer
tacgaggtgtcgcgggggt

SEQ ID NO: 99 - DNA, 19 bp, Artificial Sequence
  Sense primer
agctaacgcactcagtatc

SEQ ID NO: 100 - DNA, 20 bp, Artificial Sequence
  Sense primer
acgatggatacgaggtgtcg

SEQ ID NO: 101 - DNA, 20 bp, Artificial Sequence
  Sense primer
gtgccggagctaacgcactc

SEQ ID NO: 102 - DNA, 20 bp, Artificial Sequence
  Sense primer
aggtgcatggaattcctggt

SEQ ID NO: 103 - DNA, 20 bp, Artificial Sequence
  Sense primer
tgcatggaattcctggtgga

SEQ ID NO: 104 - DNA, 19 bp, Artificial Sequence
  Antisense primer
cagtgcaccgccttcgcca

SEQ ID NO: 105 - DNA, 19 bp, Artificial Sequence
  Antisense primer
ggccagtgcaccgccttcg

SEQ ID NO: 106 - DNA, 19 bp, Artificial Sequence
  Antisense primer
ggtggacccccgcgacacc

SEQ ID NO: 107 - DNA, 19 bp, Artificial Sequence
  Antisense primer
ggtcctcgtcccgacagac

SEQ ID NO: 108 - DNA, 20 bp, Artificial Sequence
  Antisense primer
catgcacctctcctgccctc

SEQ ID NO: 109 - DNA, 20 bp, Artificial Sequence
  Antisense primer
ttagctccggcaccgcgagg

SEQ ID NO: 110 - DNA, 19 bp, Artificial Sequence
  Antisense primer
gcgaggtggacccccgcga

SEQ ID NO: 111 - DNA, 19 bp, Artificial Sequence
  Antisense primer
tgcaccgccttcgccaccg

SEQ ID NO: 112 - DNA, 20 bp, Artificial Sequence
  Antisense primer
cgtatccatcgtttacggcg

SEQ ID NO: 113 - DNA, 20 bp, Artificial Sequence
  Antisense primer
gacccccgcgacacctcgta

SEQ ID NO: 114 - DNA, 20 bp, Artificial Sequence
  Antisense primer
gagtgcgttagctccggcac

SEQ ID NO: 115 - DNA, 19 bp, Artificial Sequence
  Antisense primer
tccaccaggaattccatgc

SEQ ID NO: 116 - DNA, 19 bp, Artificial Sequence
  Antisense primer
gccaggccagtgcaccgcc

SEQ ID NO: 117 - DNA, 20 bp, Artificial Sequence
  Antisense primer
ccaggaattccatgcacctc

SEQ ID NO: 118 - DNA, 20 bp, Artificial Sequence
  Antisense primer
cctcgtatccatcgtttacg

SEQ ID NO: 119 - DNA, 20 bp, Artificial Sequence
  Antisense primer
actgagtgcgttagctccgg

SEQ ID NO: 120 - DNA, 20 bp, Artificial Sequence
  Antisense primer
gatactgagtgcgttagctc

SEQ ID NO: 121 - DNA, 20 bp, Artificial Sequence
  Antisense primer
gcgacacctcgtatccatcg

SEQ ID NO: 122 - DNA, 20 bp, Artificial Sequence
  Antisense primer
cgggatactgagtgcgttag

SEQ ID NO: 123 - DNA, 19 bp, Artificial Sequence
  Antisense primer
gcccgatatcccccactca

SEQ ID NO: 124 - DNA, 19 bp, Artificial Sequence
  Sense primer
cagtgcaccgccttcgcca

SEQ ID NO: 125 - DNA, 19 bp, Artificial Sequence
  Sense primer
ggccagtgcaccgccttcg

SEQ ID NO: 126 - DNA, 19 bp, Artificial Sequence
  Sense primer
ggtggacccccgcgacacc

SEQ ID NO: 127 - DNA, 19 bp, Artificial Sequence
  Sense primer
ggtcctcgtcccgacagac

SEQ ID NO: 128 - DNA, 20 bp, Artificial Sequence
  Sense primer
catgcacctctcctgccctc

SEQ ID NO: 129 - DNA, 20 bp, Artificial Sequence
  Sense primer
ttagctccggcaccgcgagg

SEQ ID NO: 130 - DNA, 19 bp, Artificial Sequence
  Sense primer
gcgaggtggacccccgcga

SEQ ID NO: 131 - DNA, 19 bp, Artificial Sequence
  Sense primer
tgcaccgccttcgccaccg

SEQ ID NO: 132 - DNA, 20 bp, Artificial Sequence
  Sense primer
cgtatccatcgtttacggcg

SEQ ID NO: 133 - DNA, 20 bp, Artificial Sequence
  Sense primer
gacccccgcgacacctcgta

SEQ ID NO: 134 - DNA, 20 bp, Artificial Sequence
  Sense primer
gagtgcgttagctccggcac

SEQ ID NO: 135 - DNA, 19 bp, Artificial Sequence
  Sense primer
tccaccaggaattccatgc

SEQ ID NO: 136 - DNA, 19 bp, Artificial Sequence
  Sense primer
gccaggccagtgcaccgcc

SEQ ID NO: 137 - DNA, 20 bp, Artificial Sequence
  Sense primer
ccaggaattccatgcacctc

SEQ ID NO: 138 - DNA, 20 bp, Artificial Sequence
  Sense primer
cctcgtatccatcgtttacg

SEQ ID NO: 139 - DNA, 20 bp, Artificial Sequence
  Sense primer
actgagtgcgttagctccgg

SEQ ID NO: 140 - DNA, 20 bp, Artificial Sequence
  Sense primer
gatactgagtgcgttagctc

SEQ ID NO: 141 - DNA, 20 bp, Artificial Sequence
  Sense primer
gcgacacctcgtatccatcg

SEQ ID NO: 142 - DNA, 20 bp, Artificial Sequence
  Sense primer
cgggatactgagtgcgttag

SEQ ID NO: 143 - DNA, 19 bp, Artificial Sequence
  Sense primer
gcccgatatcccccactca

SEQ ID NO: 144 - DNA, 19 bp, Artificial Sequence
  Antisense primer
cagtgcaccgccttcgcca

SEQ ID NO: 145 - DNA, 19 bp, Artificial Sequence
  Antisense primer
ggccagtgcaccgccttcg

SEQ ID NO: 146 - DNA, 19 bp, Artificial Sequence
  Antisense primer
ggtggacccccgcgacacc

SEQ ID NO: 147 - DNA, 19 bp, Artificial Sequence
  Antisense primer
ggtcctcgtcccgacagac

SEQ ID NO: 148 - DNA, 20 bp, Artificial Sequence
  Antisense primer
catgcacctctcctgccctc

SEQ ID NO: 149 - DNA, 20 bp, Artificial Sequence
  Antisense primer
ttagctccggcaccgcgagg

SEQ ID NO: 150 - DNA, 19 bp, Artificial Sequence
  Antisense primer
gcgaggtggacccccgcga

SEQ ID NO: 151 - DNA, 19 bp, Artificial Sequence
  Antisense primer
tgcaccgccttcgccaccg

SEQ ID NO: 152 - DNA, 20 bp, Artificial Sequence
  Antisense primer
cgtatccatcgtttacggcg

SEQ ID NO: 153 - DNA, 20 bp, Artificial Sequence
  Antisense primer
gacccccgcgacacctcgta

SEQ ID NO: 154 - DNA, 20 bp, Artificial Sequence
  Antisense primer
gagtgcgttagctccggcac

SEQ ID NO: 155 - DNA, 19 bp, Artificial Sequence
  Antisense primer
tccaccaggaattccatgc

SEQ ID NO: 156 - DNA, 19 bp, Artificial Sequence
  Antisense primer
gccaggccagtgcaccgcc

SEQ ID NO: 157 - DNA, 20 bp, Artificial Sequence
  Antisense primer
ccaggaattccatgcacctc

SEQ ID NO: 158 - DNA, 20 bp, Artificial Sequence
  Antisense primer
cctcgtatccatcgtttacg

SEQ ID NO: 159 - DNA, 20 bp, Artificial Sequence
  Antisense primer
actgagtgcgttagctccgg

SEQ ID NO: 160 - DNA, 20 bp, Artificial Sequence
  Antisense primer
gatactgagtgcgttagctc

SEQ ID NO: 161 - DNA, 20 bp, Artificial Sequence
  Antisense primer
gcgacacctcgtatccatcg

SEQ ID NO: 162 - DNA, 20 bp, Artificial Sequence
  Antisense primer
cgggatactgagtgcgttag

SEQ ID NO: 163 - DNA, 19 bp, Artificial Sequence
  Antisense primer
gcccgatatcccccactca

SEQ ID NO: 164 - DNA, 20 bp, Artificial Sequence
  Sense primer
gggagacgaaaaggtaatcg

SEQ ID NO: 165 - DNA, 20 bp, Artificial Sequence
  Sense primer
aaagttctttcggtgacggg

SEQ ID NO: 166 - DNA, 20 bp, Artificial Sequence
  Sense primer
cggggaaggttgatatgtta

SEQ ID NO: 167 - DNA, 19 bp, Artificial Sequence
  Sense primer
gagggagaaaccgggggat

SEQ ID NO: 168 - DNA, 19 bp, Artificial Sequence
  Sense primer
aatcgctaatatcggttac

SEQ ID NO: 169 - DNA, 20 bp, Artificial Sequence
  Sense primer
ccgggggatcttcggacctc

SEQ ID NO: 170 - DNA, 20 bp, Artificial Sequence
  Sense primer
taatatcgcctgctgttgac

SEQ ID NO: 171 - DNA, 19 bp, Artificial Sequence
  Sense primer
tcggtgacggggaaggttg

SEQ ID NO: 172 - DNA, 19 bp, Artificial Sequence
  Sense primer
ggagaaaccgggggatctt

SEQ ID NO: 173 - DNA, 20 bp, Artificial Sequence
  Sense primer
acgtcctgagggagaaaccg

SEQ ID NO: 174 - DNA, 20 bp, Artificial Sequence
  Sense primer
agacgaaaaggtaatcgcta

SEQ ID NO: 175 - DNA, 19 bp, Artificial Sequence
  Sense primer
gtgacggggaaggttgata

SEQ ID NO: 176 - DNA, 19 bp, Artificial Sequence
  Sense primer
gaaaccgggggatcttcgg

SEQ ID NO: 177 - DNA, 20 bp, Artificial Sequence
  Sense primer
tcctgagggagaaaccgggg

SEQ ID NO: 178 - DNA, 20 bp, Artificial Sequence
  Sense primer
cgaaaaggtaatcgctaata

SEQ ID NO: 179 - DNA, 20 bp, Artificial Sequence
  Sense primer
aaaggtaatcgctaatatcg

SEQ ID NO: 180 - DNA, 20 bp, Artificial Sequence
  Sense primer
tcgtgggagacgaaaaggta

SEQ ID NO: 181 - DNA, 20 bp, Artificial Sequence
  Sense primer
cggacctcgtgctattggag

SEQ ID NO: 182 - DNA, 19 bp, Artificial Sequence
  Sense primer
gttctttcggtgacgggga

SEQ ID NO: 183 - DNA, 19 bp, Artificial Sequence
  Sense primer
ctttcggtgacggggaagg

SEQ ID NO: 184 - DNA, 19 bp, Artificial Sequence
  Antisense primer
atcccccggtttctccctc

SEQ ID NO: 185 - DNA, 19 bp, Artificial Sequence
  Antisense primer
atattagcgattacctttt

SEQ ID NO: 186 - DNA, 20 bp, Artificial Sequence
  Antisense primer
caaccttccccgtcaccgaa

SEQ ID NO: 187 - DNA, 20 bp, Artificial Sequence
  Antisense primer
ccgaagatcccccggtttct

SEQ ID NO: 188 - DNA, 20 bp, Artificial Sequence
  Antisense primer
ctccaatagcacgaggtccg

SEQ ID NO: 189 - DNA, 20 bp, Artificial Sequence
  Antisense primer
accgatattagcgattacct

SEQ ID NO: 190 - DNA, 19 bp, Artificial Sequence
  Antisense primer
aagatcccccggtttctcc

SEQ ID NO: 191 - DNA, 20 bp, Artificial Sequence
  Antisense primer
tatcaaccttccccgtcacc

SEQ ID NO: 192 - DNA, 20 bp, Artificial Sequence
  Antisense primer
ggtttctccctcaggacgta

SEQ ID NO: 193 - DNA, 20 bp, Artificial Sequence
  Antisense primer
ggtccgaagatcccccggtt

SEQ ID NO: 194 - DNA, 19 bp, Artificial Sequence
  Antisense primer
tttcacgacagacctaatg

SEQ ID NO: 195 - DNA, 20 bp, Artificial Sequence
  Antisense primer
gtaaccgatattagcgatta

SEQ ID NO: 196 - DNA, 20 bp, Artificial Sequence
  Antisense primer
acatatcaaccttccccgtc

SEQ ID NO: 197 - DNA, 20 bp, Artificial Sequence
  Antisense primer
cccggtttctccctcaggac

SEQ ID NO: 198 - DNA, 20 bp, Artificial Sequence
  Antisense primer
gcgattaccttttcgtctcc

SEQ ID NO: 199 - DNA, 20 bp, Artificial Sequence
  Antisense primer
ccccgtcaccgaaagaactt

SEQ ID NO: 200 - DNA, 20 bp, Artificial Sequence
  Antisense primer
ttaacatatcaaccttcccc

SEQ ID NO: 201 - DNA, 20 bp, Artificial Sequence
  Antisense primer
ttagcgattaccttttcgtc

SEQ ID NO: 202 - DNA, 20 bp, Artificial Sequence
  Antisense primer
cttccccgtcaccgaaagaa

SEQ ID NO: 203 - DNA, 20 bp, Artificial Sequence
  Antisense primer
attaccttttcgtctcccac

SEQ ID NO: 204 - DNA, 19 bp, Artificial Sequence
  Sense primer
gggaaaccgtgagggcgct

SEQ ID NO: 205 - DNA, 20 bp, Artificial Sequence
  Sense primer
gcgaaacgtccccaatgcgg

SEQ ID NO: 206 - DNA, 21 bp, Artificial Sequence
  Sense primer
ccgcagggaaaccggtaagcc

SEQ ID NO: 207 - DNA, 20 bp, Artificial Sequence
  Sense primer
cccgggaaagggcagtgata

SEQ ID NO: 208 - DNA, 19 bp, Artificial Sequence
  Sense primer
gggaaagggcagtgatact

SEQ ID NO: 209 - DNA, 20 bp, Artificial Sequence
  Sense primer
aatccggggcaggcgaaggg

SEQ ID NO: 210 - DNA, 20 bp, Artificial Sequence
  Sense primer
agggtactggaacgtccctt

SEQ ID NO: 211 - DNA, 21 bp, Artificial Sequence
  Sense primer
aagcgtccggccagaacgcgc

SEQ ID NO: 212 - DNA, 20 bp, Artificial Sequence
  Sense primer
cgcctaaaggggcatgggct

SEQ ID NO: 213 - DNA, 20 bp, Artificial Sequence
  Sense primer
ggctatttcccgctcatgcc

SEQ ID NO: 214 - DNA, 21 bp, Artificial Sequence
  Sense primer
cgtacgccctcgggtaagagg

SEQ ID NO: 215 - DNA, 20 bp, Artificial Sequence
  Sense primer
aacggcccgccaaaccgata

SEQ ID NO: 216 - DNA, 19 bp, Artificial Sequence
  Sense primer
agccggccctgcaagtcac

SEQ ID NO: 217 - DNA, 19 bp, Artificial Sequence
  Sense primer
cactgcttaaagacccggg

SEQ ID NO: 218 - DNA, 21 bp, Artificial Sequence
  Sense primer
ggagctaatccggggcaggcg

SEQ ID NO: 219 - DNA, 20 bp, Artificial Sequence
  Sense primer
aaaccgtgagggcgctaccc

SEQ ID NO: 220 - DNA, 21 bp, Artificial Sequence
  Sense primer
aggcgaagggtactggaacgt

SEQ ID NO: 221 - DNA, 20 bp, Artificial Sequence
  Sense primer
acccccagtgctcccgaaag

SEQ ID NO: 222 - DNA, 21 bp, Artificial Sequence
  Sense primer
cccttcgcctaaaggggcatg

SEQ ID NO: 223 - DNA, 21 bp, Artificial Sequence
  Sense primer
gcatgggctatttcccgctca

SEQ ID NO: 224 - DNA, 19 bp, Artificial Sequence
  Sense primer
gggaaaccgtgagggcgct

SEQ ID NO: 225 - DNA, 20 bp, Artificial Sequence
  Sense primer
gcgaaacgtccccaatgcgg

SEQ ID NO: 226 - DNA, 21 bp, Artificial Sequence
  Antisense primer
ccgcattggggacgtttcgcg

SEQ ID NO: 227 - DNA, 21 bp, Artificial Sequence
  Antisense primer
gcgccctcacggtttcccgca

SEQ ID NO: 228 - DNA, 21 bp, Artificial Sequence
  Antisense primer
ccgcattggggacgtttcgcg

SEQ ID NO: 229 - DNA, 21 bp, Artificial Sequence
  Antisense primer
gcgccctcacggtttcccgca

SEQ ID NO: 230 - DNA, 21 bp, Artificial Sequence
  Antisense primer
ttcccgcattggggacgtttc

SEQ ID NO: 231 - DNA, 20 bp, Artificial Sequence
  Antisense primer
tagcgccctcacggtttccc

SEQ ID NO: 232 - DNA, 20 bp, Artificial Sequence
  Antisense primer
ggcttaccggtttccctgcg

SEQ ID NO: 233 - DNA, 19 bp, Artificial Sequence
  Antisense primer
ctgccctttcccgggttga

SEQ ID NO: 234 - DNA, 19 bp, Artificial Sequence
  Antisense primer
tcactgccctttcccgggt

SEQ ID NO: 235 - DNA, 19 bp, Artificial Sequence
  Antisense primer
gtatcactgccctttcccg

SEQ ID NO: 236 - DNA, 20 bp, Artificial Sequence
  Antisense primer
gcccgggtctttaagcagtg

SEQ ID NO: 237 - DNA, 21 bp, Artificial Sequence
  Antisense primer
ctcccgccccctagccctgca

SEQ ID NO: 238 - DNA, 21 bp, Artificial Sequence
  Antisense primer
cccgggatctgtggatttcgc

SEQ ID NO: 239 - DNA, 19 bp, Artificial Sequence
  Antisense primer
tacccgagggcgtacgact

SEQ ID NO: 240 - DNA, 21 bp, Artificial Sequence
  Antisense primer
cctcttacccgagggcgtacg

SEQ ID NO: 241 - DNA, 19 bp, Artificial Sequence
  Antisense primer
ttcgcctgccccggattag

SEQ ID NO: 242 - DNA, 21 bp, Artificial Sequence
  Antisense primer
ggcggcaggcttaccggtttc

SEQ ID NO: 243 - DNA, 21 bp, Artificial Sequence
  Antisense primer
cggattagctccagtttcccg

SEQ ID NO: 244 - DNA, 19 bp, Artificial Sequence
  Antisense primer
ggacgttccagtacccttc

SEQ ID NO: 245 - DNA, 20 bp, Artificial Sequence
  Antisense primer
ccccggattagctccagttt

SEQ ID NO: 246 - DNA, 21 bp, Artificial Sequence
  Antisense primer
tacccttcgcctgccccggat

SEQ ID NO: 247 - DNA, 19 bp, Artificial Sequence
  Antisense primer
ccatgcccctttaggcgaa

SEQ ID NO: 248 - DNA, 21 bp, Artificial Sequence
  Sense primer
agagtcaacctgacgagctta

SEQ ID NO: 249 - DNA, 21 bp, Artificial Sequence
  Sense primer
gtcaacctgacgagcttactc

SEQ ID NO: 250 - DNA, 20 bp, Artificial Sequence
  Sense primer
tgagagtcaacctgacgagc

SEQ ID NO: 251 - DNA, 21 bp, Artificial Sequence
  Sense primer
gagcttactcgatagcaggag

SEQ ID NO: 252 - DNA, 19 bp, Artificial Sequence
  Sense primer
tttaattcgagagggttaa

SEQ ID NO: 253 - DNA, 21 bp, Artificial Sequence
  Sense primer
cttactcgatagcaggagagg

SEQ ID NO: 254 - DNA, 21 bp, Artificial Sequence
  Sense primer
aatcaaatctgatgtcggtga

SEQ ID NO: 255 - DNA, 19 bp, Artificial Sequence
  Sense primer
ggttaaatcaaatctgatg

SEQ ID NO: 256 - DNA, 21 bp, Artificial Sequence
  Sense primer
ttcgagagggttaaatcaaat

SEQ ID NO: 257 - DNA, 21 bp, Artificial Sequence
  Sense primer
caaatctgatgtcggtgagga

SEQ ID NO: 258 - DNA, 19 bp, Artificial Sequence
  Sense primer
taaatcaaatctgatgtcg

SEQ ID NO: 259 - DNA, 21 bp, Artificial Sequence
  Sense primer
gagagggttaaatcaaatctg

SEQ ID NO: 260 - DNA, 21 bp, Artificial Sequence
  Sense primer
atctgatgtcggtgaggaggg

SEQ ID NO: 261 - DNA, 19 bp, Artificial Sequence
  Sense primer
aattcgagagggttaaatc

SEQ ID NO: 262 - DNA, 19 bp, Artificial Sequence
  Sense primer
gatgtcggtgaggagggtt

SEQ ID NO: 263 - DNA, 19 bp, Artificial Sequence
  Sense primer
gagggatggcagtgtcgga

SEQ ID NO: 264 - DNA, 19 bp, Artificial Sequence
  Sense primer
tggccaagacttttctcat

SEQ ID NO: 265 - DNA, 20 bp, Artificial Sequence
  Sense primer
gatgagtctgcaacctatca

SEQ ID NO: 266 - DNA, 21 bp, Artificial Sequence
  Sense primer
tagcagagaggtggtgcatgg

SEQ ID NO: 267 - DNA, 21 bp, Artificial Sequence
  Sense primer
acggccactgctatcaagttc

SEQ ID NO: 268 - DNA, 20 bp, Artificial Sequence
  Antisense primer
aagctcgtcaggttgactct

SEQ ID NO: 269 - DNA, 19 bp, Artificial Sequence
  Antisense primer
gtaagctcgtcaggttgac

SEQ ID NO: 270 - DNA, 19 bp, Artificial Sequence
  Antisense primer
cgagtaagctcgtcaggtt

SEQ ID NO: 271 - DNA, 19 bp, Artificial Sequence
  Antisense primer
ctgctatcgagtaagctcg

SEQ ID NO: 272 - DNA, 19 bp, Artificial Sequence
  Antisense primer
tttaaccctctcgaattaa

SEQ ID NO: 273 - DNA, 19 bp, Artificial Sequence
  Antisense primer
ctcctgctatcgagtaagc

SEQ ID NO: 274 - DNA, 20 bp, Artificial Sequence
  Antisense primer
tcagatttgatttaaccctc

SEQ ID NO: 275 - DNA, 20 bp, Artificial Sequence
  Antisense primer
accctcctcaccgacatcag

SEQ ID NO: 276 - DNA, 19 bp, Artificial Sequence
  Antisense primer
acatcagatttgatttaac

SEQ ID NO: 277 - DNA, 19 bp, Artificial Sequence
  Antisense primer
ccgacatcagatttgattt

SEQ ID NO: 278 - DNA, 19 bp, Artificial Sequence
  Antisense primer
tgatttaaccctctcgaat

SEQ ID NO: 279 - DNA, 19 bp, Artificial Sequence
  Antisense primer
tcaccgacatcagatttga

SEQ ID NO: 280 - DNA, 19 bp, Artificial Sequence
  Antisense primer
atttgatttaaccctctcg

SEQ ID NO: 281 - DNA, 21 bp, Artificial Sequence
  Antisense primer
ctacctgataggttgcagact

SEQ ID NO: 282 - DNA, 20 bp, Artificial Sequence
  Antisense primer
gcaccacctctctgctatcg

SEQ ID NO: 283 - DNA, 19 bp, Artificial Sequence
  Antisense primer
atccctcaacggaaaagca

SEQ ID NO: 284 - DNA, 20 bp, Artificial Sequence
  Antisense primer
acacttaaagtgaacgccct

SEQ ID NO: 285 - DNA, 19 bp, Artificial Sequence
  Antisense primer
tcgctccgacactgccatc

SEQ ID NO: 286 - DNA, 20 bp, Artificial Sequence
  Antisense primer
ccgatctcatgtcttgcagt

SEQ ID NO: 287 - DNA, 19 bp, Artificial Sequence
  Antisense primer
atgagaaaagtcttggcca

SEQ ID NO: 288 - DNA, 19 bp, Artificial Sequence
  Sense primer
agggcgttacccctagtgc

SEQ ID NO: 289 - DNA, 19 bp, Artificial Sequence
  Sense primer
tacccctagtgccctcgca

SEQ ID NO: 290 - DNA, 21 bp, Artificial Sequence
  Sense primer
gcgcccgtagccggcctgtaa

SEQ ID NO: 291 - DNA, 21 bp, Artificial Sequence
  Sense primer
gagcttctcctccgcgagggg

SEQ ID NO: 292 - DNA, 21 bp, Artificial Sequence
  Sense primer
gcaccaggcgcggaacgtccc

SEQ ID NO: 293 - DNA, 20 bp, Artificial Sequence
  Sense primer
gaggtcgagcttctcctccg

SEQ ID NO: 294 - DNA, 19 bp, Artificial Sequence
  Sense primer
ccctagtgccctcgcaaga

SEQ ID NO: 295 - DNA, 21 bp, Artificial Sequence
  Sense primer
cccgtagccggcctgtaaagt

SEQ ID NO: 296 - DNA, 21 bp, Artificial Sequence
  Sense primer
cggggtgggaggtcgagcttc

SEQ ID NO: 297 - DNA, 20 bp, Artificial Sequence
  Sense primer
gtcgagcttctcctccgcga

SEQ ID NO: 298 - DNA, 21 bp, Artificial Sequence
  Sense primer
ggtgggaggtcgagcttctcc

SEQ ID NO: 299 - DNA, 19 bp, Artificial Sequence
  Sense primer
tcggggtgggaggtcgagc

SEQ ID NO: 300 - DNA, 19 bp, Artificial Sequence
  Sense primer
gcgttacccctagtgccct

SEQ ID NO: 301 - DNA, 21 bp, Artificial Sequence
  Sense primer
taggggtagggctaagccatg

SEQ ID NO: 302 - DNA, 19 bp, Artificial Sequence
  Sense primer
cgcaccaggcgcggaacgt

SEQ ID NO: 303 - DNA, 19 bp, Artificial Sequence
  Sense primer
gggaggtcgagcttctcct

SEQ ID NO: 304 - DNA, 20 bp, Artificial Sequence
  Sense primer
aggtggaggaataagcgggg

SEQ ID NO: 305 - DNA, 19 bp, Artificial Sequence
  Sense primer
gaaaggtggaggaataagc

SEQ ID NO: 306 - DNA, 21 bp, Artificial Sequence
  Sense primer
gggagtcgtacgctctcggga

SEQ ID NO: 307 - DNA, 21 bp, Artificial Sequence
  Sense primer
ctaacctgcccttgggatctg

SEQ ID NO: 308 - DNA, 19 bp, Artificial Sequence
  Antisense primer
ggcactaggggtaacgccc

SEQ ID NO: 309 - DNA, 20 bp, Artificial Sequence
  Antisense primer
agaagctcgacctcccaccc

SEQ ID NO: 310 - DNA, 20 bp, Artificial Sequence
  Antisense primer
tacaggccggctacgggcgc

SEQ ID NO: 311 - DNA, 19 bp, Artificial Sequence
  Antisense primer
agctcgacctcccaccccg

SEQ ID NO: 312 - DNA, 19 bp, Artificial Sequence
  Antisense primer
cccctcgcggaggagaagc

SEQ ID NO: 313 - DNA, 19 bp, Artificial Sequence
  Antisense primer
tgcgagggcactaggggta

SEQ ID NO: 314 - DNA, 21 bp, Artificial Sequence
  Antisense primer
tgactttacaggccggctacg

SEQ ID NO: 315 - DNA, 21 bp, Artificial Sequence
  Antisense primer
catggcttagccctaccccta

SEQ ID NO: 316 - DNA, 20 bp, Artificial Sequence
  Antisense primer
aggagaagctcgacctccca

SEQ ID NO: 317 - DNA, 19 bp, Artificial Sequence
  Antisense primer
gacgttccgcgcctggtgc

SEQ ID NO: 318 - DNA, 20 bp, Artificial Sequence
  Antisense primer
ctttacaggccggctacggg

SEQ ID NO: 319 - DNA, 19 bp, Artificial Sequence
  Antisense primer
tcttgcgagggcactaggg

SEQ ID NO: 320 - DNA, 20 bp, Artificial Sequence
  Antisense primer
cggaggagaagctcgacctc

SEQ ID NO: 321 - DNA, 20 bp, Artificial Sequence
  Antisense primer
tcgcggaggagaagctcgac

SEQ ID NO: 322 - DNA, 19 bp, Artificial Sequence
  Antisense primer
gagggcactaggggtaacg

SEQ ID NO: 323 - DNA, 20 bp, Artificial Sequence
  Antisense primer
accccgaggggcaagaggcc

SEQ ID NO: 324 - DNA, 21 bp, Artificial Sequence
  Antisense primer
ggggttatccagatcccaagg

SEQ ID NO: 325 - DNA, 20 bp, Artificial Sequence
  Antisense primer
gccacgccctcttcccgaga

SEQ ID NO: 326 - DNA, 20 bp, Artificial Sequence
  Antisense primer
gttatccagatcccaagggc

SEQ ID NO: 327 - DNA, 21 bp, Artificial Sequence
  Antisense primer
cttattcctccacctttctgg

SEQ ID NO: 328 - DNA, 19 bp, Artificial Sequence
  Sense primer
taaaccctgccgcagttgg

SEQ ID NO: 329 - DNA, 19 bp, Artificial Sequence
  Sense primer
ccttaaaccctgccgcagt

SEQ ID NO: 330 - DNA, 19 bp, Artificial Sequence
  Sense primer
gtcctggaacggttcctcg

SEQ ID NO: 331 - DNA, 21 bp, Artificial Sequence
  Sense primer
ctctacaaaggcgggggaata

SEQ ID NO: 332 - DNA, 20 bp, Artificial Sequence
  Sense primer
ctggaacggttcctcgctga

SEQ ID NO: 333 - DNA, 21 bp, Artificial Sequence
  Sense primer
ggcgaggagtcctggaacggt

SEQ ID NO: 334 - DNA, 19 bp, Artificial Sequence
  Sense primer
tttccccgctctacaaagg

SEQ ID NO: 335 - DNA, 21 bp, Artificial Sequence
  Sense primer
tacaaaggcgggggaataagc

SEQ ID NO: 336 - DNA, 19 bp, Artificial Sequence
  Sense primer
cgctctacaaaggcggggg

SEQ ID NO: 337 - DNA, 20 bp, Artificial Sequence
  Sense primer
ataggcgaggagtcctggaa

SEQ ID NO: 338 - DNA, 19 bp, Artificial Sequence
  Sense primer
ccataggcgaggagtcctg

SEQ ID NO: 339 - DNA, 19 bp, Artificial Sequence
  Sense primer
gcttttccccgctctacaa

SEQ ID NO: 340 - DNA, 20 bp, Artificial Sequence
  Sense primer
gctaacctaccctgaggagg

SEQ ID NO: 341 - DNA, 20 bp, Artificial Sequence
  Sense primer
tctcccataggcgaggagtc

SEQ ID NO: 342 - DNA, 19 bp, Artificial Sequence
  Sense primer
tggctaacctaccctgagg

SEQ ID NO: 343 - DNA, 19 bp, Artificial Sequence
  Sense primer
ataatctcccataggcgag

SEQ ID NO: 344 - DNA, 20 bp, Artificial Sequence
  Sense primer
tgaggagggagataaccccg

SEQ ID NO: 345 - DNA, 21 bp, Artificial Sequence
  Sense primer
acacgtggctaacctaccctg

SEQ ID NO: 346 - DNA, 19 bp, Artificial Sequence
  Sense primer
cctgaggagggagataacc

SEQ ID NO: 347 - DNA, 19 bp, Artificial Sequence
  Sense primer
aaactggggataatctccc

SEQ ID NO: 348 - DNA, 19 bp, Artificial Sequence
  Antisense primer
ccaactgcggcagggttta

SEQ ID NO: 349 - DNA, 19 bp, Artificial Sequence
  Antisense primer
actgcggcagggtttaagg

SEQ ID NO: 350 - DNA, 21 bp, Artificial Sequence
  Antisense primer
cgaggaaccgttccaggactc

SEQ ID NO: 351 - DNA, 20 bp, Artificial Sequence
  Antisense primer
aaccgttccaggactcctcg

SEQ ID NO: 352 - DNA, 21 bp, Artificial Sequence
  Antisense primer
tccaggactcctcgcctatgg

SEQ ID NO: 353 - DNA, 19 bp, Artificial Sequence
  Antisense primer
cctttgtagagcggggaaa

SEQ ID NO: 354 - DNA, 20 bp, Artificial Sequence
  Antisense primer
agcgaggaaccgttccagga

SEQ ID NO: 355 - DNA, 21 bp, Artificial Sequence
  Antisense primer
cgttccaggactcctcgccta

SEQ ID NO: 356 - DNA, 19 bp, Artificial Sequence
  Antisense primer
cccccgcctttgtagagcg

SEQ ID NO: 357 - DNA, 20 bp, Artificial Sequence
  Antisense primer
ttcagcgaggaaccgttcca

SEQ ID NO: 358 - DNA, 19 bp, Artificial Sequence
  Antisense primer
attcccccgcctttgtaga

SEQ ID NO: 359 - DNA, 19 bp, Artificial Sequence
  Antisense primer
ttgtagagcggggaaaagc

SEQ ID NO: 360 - DNA, 21 bp, Artificial Sequence
  Antisense primer
atctccctcctcagggtaggt

SEQ ID NO: 361 - DNA, 19 bp, Artificial Sequence
  Antisense primer
gggttatctccctcctcag

SEQ ID NO: 362 - DNA, 19 bp, Artificial Sequence
  Antisense primer
tcgcctatgggagattatc

SEQ ID NO: 363 - DNA, 20 bp, Artificial Sequence
  Antisense primer
tcagggtaggttagccacgt

SEQ ID NO: 364 - DNA, 19 bp, Artificial Sequence
  Antisense primer
cctcagggtaggttagcca

SEQ ID NO: 365 - DNA, 19 bp, Artificial Sequence
  Antisense primer
ccggggttatctccctcct

SEQ ID NO: 366 - DNA, 19 bp, Artificial Sequence
  Antisense primer
tcctcgcctatgggagatt

SEQ ID NO: 367 - DNA, 19 bp, Artificial Sequence
  Antisense primer
cctcctcagggtaggttag

SEQ ID NO: 368 - DNA, 20 bp, Artificial Sequence
  Sense primer
tcctgaaaggacgaccggtg

SEQ ID NO: 369 - DNA, 19 bp, Artificial Sequence
  Sense primer
ggactgagggctgtaactc

SEQ ID NO: 370 - DNA, 21 bp, Artificial Sequence
  Sense primer
gaggttgaatgtactttcagg

SEQ ID NO: 371 - DNA, 20 bp, Artificial Sequence
  Sense primer
ggtggcgaaagcgttcaact

SEQ ID NO: 372 - DNA, 19 bp, Artificial Sequence
  Sense primer
gccctcacgaatgtggatt

SEQ ID NO: 373 - DNA, 20 bp, Artificial Sequence
  Sense primer
acctcgaaacccgttcgtag

SEQ ID NO: 374 - DNA, 19 bp, Artificial Sequence
  Sense primer
tccgtagtaatcgtaggtc

SEQ ID NO: 375 - DNA, 21 bp, Artificial Sequence
  Sense primer
atcctgtaatcctgaaaggac

SEQ ID NO: 376 - DNA, 20 bp, Artificial Sequence
  Sense primer
gtagtcaggactgagggctg

SEQ ID NO: 377 - DNA, 21 bp, Artificial Sequence
  Sense primer
aggacgaccggtggcgaaagc

SEQ ID NO: 378 - DNA, 20 bp, Artificial Sequence
  Sense primer
taactcgccctcacgaatgt

SEQ ID NO: 379 - DNA, 19 bp, Artificial Sequence
  Sense primer
gaaggtgttaagtgggtca

SEQ ID NO: 380 - DNA, 21 bp, Artificial Sequence
  Sense primer
aaacccgttcgtagtcaggac

SEQ ID NO: 381 - DNA, 20 bp, Artificial Sequence
  Sense primer
tacggtgaatatgcccctgc

SEQ ID NO: 382 - DNA, 21 bp, Artificial Sequence
  Sense primer
cacttggtgttgcttctccgt

SEQ ID NO: 383 - DNA, 19 bp, Artificial Sequence
  Sense primer
gatcacttttattgagtct

SEQ ID NO: 384 - DNA, 20 bp, Artificial Sequence
  Sense primer
agcatcaggaataagggctg

SEQ ID NO: 385 - DNA, 19 bp, Artificial Sequence
  Sense primer
aagacccccatctctaatt

SEQ ID NO: 386 - DNA, 19 bp, Artificial Sequence
  Sense primer
ccggtcttataaatcttca

SEQ ID NO: 387 - DNA, 20 bp, Artificial Sequence
  Sense primer
ataacgagcaagacccccat

SEQ ID NO: 388 - DNA, 20 bp, Artificial Sequence
  Antisense primer
caggggcatattcaccgtag

SEQ ID NO: 389 - DNA, 19 bp, Artificial Sequence
  Antisense primer
tcaggattacaggatttta

SEQ ID NO: 390 - DNA, 21 bp, Artificial Sequence
  Antisense primer
accctgaaagtacattcaacc

SEQ ID NO: 391 - DNA, 19 bp, Artificial Sequence
  Antisense primer
gccaccggtcgtcctttca

SEQ ID NO: 392 - DNA, 19 bp, Artificial Sequence
  Antisense primer
ctagttgaacgctttcgcc

SEQ ID NO: 393 - DNA, 21 bp, Artificial Sequence
  Antisense primer
tcgtcctttcaggattacagg

SEQ ID NO: 394 - DNA, 21 bp, Artificial Sequence
  Antisense primer
acgctttcgccaccggtcgtc

SEQ ID NO: 395 - DNA, 19 bp, Artificial Sequence
  Antisense primer
gggtttcgaggttagcttc

SEQ ID NO: 396 - DNA, 19 bp, Artificial Sequence
  Antisense primer
ccctcagtcctgactacga

SEQ ID NO: 397 - DNA, 20 bp, Artificial Sequence
  Antisense primer
ctgaagatttataagaccgg

SEQ ID NO: 398 - DNA, 21 bp, Artificial Sequence
  Antisense primer
ttacagccctcagtcctgact

SEQ ID NO: 399 - DNA, 21 bp, Artificial Sequence
  Antisense primer
aatccacattcgtgagggcga

SEQ ID NO: 400 - DNA, 20 bp, Artificial Sequence
  Antisense primer
atgggggtcttgctcgttat

SEQ ID NO: 401 - DNA, 19 bp, Artificial Sequence
  Antisense primer
gctgttgacctacgattac

SEQ ID NO: 402 - DNA, 21 bp, Artificial Sequence
  Antisense primer
cctacgattactacggaatcc

SEQ ID NO: 403 - DNA, 19 bp, Artificial Sequence
  Antisense primer
acccacttaacaccttcgc

SEQ ID NO: 404 - DNA, 20 bp, Artificial Sequence
  Antisense primer
cccaagtcttacagtctctt

SEQ ID NO: 405 - DNA, 20 bp, Artificial Sequence
  Antisense primer
ctaccctgaaagtacattca

SEQ ID NO: 406 - DNA, 19 bp, Artificial Sequence
  Antisense primer
cagcccttattcctgatgc

SEQ ID NO: 407 - DNA, 20 bp, Artificial Sequence
  Antisense primer
ggtcgtcctttcaggattac

SEQ ID NO: 408 - DNA, 18 bp, Artificial Sequence
  PCR primer
gtgccagcmgccgcggta

SEQ ID NO: 409 - DNA, 17 bp, Artificial Sequence
  PCR primer
cctacgggaggcagcag

SEQ ID NO: 410 - DNA, 19 bp, Artificial Sequence
  PCR primer
ccgtcaattcctttgagtt

SEQ ID NO: 411 - DNA, 18 bp, Artificial Sequence
  PCR primer
gctgcctcccgtaggagt

SEQ ID NO: 412 - DNA, 18 bp, Artificial Sequence
  PCR primer
gggcggwgtgtacaaggc

SEQ ID NO: 413 - DNA, 20 bp, Artificial Sequence
  PCR primer
acggggcgcagcaggcgcga

SEQ ID NO: 414 - DNA, 19 bp, Artificial Sequence
  PCR primer
gtgccagcagccgcggtaa

SEQ ID NO: 415 - DNA, 19 bp, Artificial Sequence
  PCR primer
yccggcgttgamtccaatt

SEQ ID NO: 416 - DNA, 20 bp, Artificial Sequence
  PCR primer
gtgctcccccgccaattcct

SEQ ID NO: 417 - DNA, 17 bp, Artificial Sequence
  PCR primer
attaccgcggctgctgg